(19) United States
(12) Patent Application Publication
(10) Pub. No.: US 2019/0221284 A1
(43) Pub. Date: Jul. 18, 2019

(54) INFORMATION PROCESSING APPARATUS, INFORMATION PROCESSING SYSTEM, INFORMATION PROCESSING METHOD, AND STORAGE MEDIUM

(71) Applicant: FUJITSU LIMITED, Kawasaki-shi (JP)

(72) Inventors: Motoyuki Kawaba, Kawasaki (JP); Yoshifumi Ujibashi, Kawasaki (JP)

(73) Assignee: FUJITSU LIMITED, Kawasaki-shi (JP)

(21) Appl. No.: 16/365,048

(22) Filed: Mar. 26, 2019

Related U.S. Application Data
(63) Continuation of application No. PCT/JP2018/000539, filed on Jan. 11, 2018.

(30) Foreign Application Priority Data
Jan. 24, 2017 (JP) 2017-010416

(51) Int. Cl. G16B 20/20; G16B 30/00

(52) U.S. Cl. CPC G16B 20/20; G16H 70/60; G16B 30/00

(57) ABSTRACT

An information processing apparatus includes a memory; and a processor coupled to the memory and configured to execute processing relating to a plurality of sequences according to a plurality of variant patterns included in each of the plurality of sequences, wherein the executing the processing relating to the plurality of sequences includes: when variant patterns at a same variant position are same among the plurality of sequences, executing processing of exclusion of the same variant patterns from the plurality of sequences, and storing the plurality of sequences for which the processing of exclusion has been executed in the memory.

### CROSS-REFERENCE TO RELATED APPLICATION

This application is a continuation application of International Application PCT/JP2018/000539, filed on Jan. 11, 2018 and designated the U.S., the entire contents of which are incorporated herein by reference. The International Application PCT/JP2018/000539 is based upon and claims the benefit of priority of the prior Japanese Patent Application No. 2017-010416, filed on Jan. 24, 2017, the entire contents of which are incorporated herein by reference.

### FIELD

The embodiments discussed herein are related to an information processing apparatus, an information processing system, an information processing method, and a storage medium.

### BACKGROUND

In genetic information, parts that cause individual differences, for example, parts at which the genetic information differs depending on the individual (these parts may be referred to as “variant”), exist at tens of millions of places. Furthermore, there is a possibility that the genetic information regarding partial variants among them has a relation to incidence of a specific disease. For this reason, researches are being advanced on techniques in which whether or not a significant difference exists in the appearance frequency of a variant pattern between a population affected by a disease of the object and an unaffected population is tested regarding each of individual variants to thereby analyze the above-described variants having a relation to the incidence of the disease and the variant patterns thereof.

“Genetic information” may be referred to as “base sequence of DNA (deoxyribonucleic acid)” or “variant information on the human genome.”

As related arts, the following documents are disclosed, for example.
(1) Japanese Laid-open Patent Publication No. 2004-166565
(2) Japanese Laid-open Patent Publication No. 2004-234104
(3) FUJITSU LABORATORIES LTD., “Genome Jouhou no Kaisekishori wo Kousokuka suru Gijutsu wo Kaihatsu (in English, technique to enhance speed of analysis processing of genome information has been developed),” [online], Mar. 15, 2016, [retrieved on Jan. 10, 2017], the Internet <URL: pr.fujitsu.com/jp/news/2016/03/15.html>

Approximately 20 million variants are included in the variant information on the human genome. For example, if one variant is represented by 2-bit information, the data amount of variant information regarding 100 thousand people is approximately 500 GB (gigabytes). If the data capacity of a primary storing apparatus of a computer used for retrieval and analysis of the variant information on the human genome is not lower than the data amount of the variant information, access to a secondary storing apparatus occurs in processing of retrieval and analysis.

As exemplified above, if the number of variant patterns included in sequence data of the processing target is large and the data amount of the sequence data is large, it is difficult to store the whole of the sequence data in the primary storing apparatus and access to the secondary storing apparatus occurs. This possibly lowers the processing speed of retrieval and analysis of the sequence data. In view of the above, it is desirable that the amount of data stored in a memory may be reduced in plural sequences each included plural variant patterns.

### SUMMARY

According to an aspect of the embodiments, an information processing apparatus includes a memory; and a processor coupled to the memory and configured to execute processing relating to a plurality of sequences according to a plurality of variant patterns included in each of the plurality of sequences, wherein the executing the processing relating to the plurality of sequences includes: when variant patterns at a same variant position are same among the plurality of sequences, executing processing of exclusion of the same variant patterns from the plurality of sequences, and storing the plurality of sequences for which the processing of exclusion has been executed in the memory.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1A and FIG. 1B are graphs illustrating one example of distribution of variant patterns in a variant without specificity and a variant with specificity;

FIG. 2 is a block diagram illustrating an outline of aggregation processing of variant information;

FIG. 3 is a diagram illustrating one example of variant information;

FIG. 4A and FIG. 4B are diagrams explaining extraction processing of variant sequences;

FIG. 5 is a diagram explaining aggregation processing of a variant sequence;

FIG. 6 is a diagram explaining aggregation processing of a variant sequence;

FIG. 7A and FIG. 7B are diagrams illustrating a genome type structure with variant master information;

FIG. 8 is a diagram explaining retrieval processing of variant information;

FIG. 9 is a block diagram illustrating a hardware configuration of an information processing system in one example of an embodiment;

FIG. 10A and FIG. 1013 are block diagrams illustrating functional configurations of an information processing apparatus and a terminal in one example of the embodiment;

FIG. 11A and FIG. 11B are diagrams illustrating a genome type structure in one example of the embodiment with variant master information;

FIG. 12 is a diagram explaining creation processing of group statistical information and group dividing information in one example of the embodiment;

FIG. 13 is a diagram explaining compression processing of uncompressed variant information in one example of the embodiment;

FIG. 14 is a diagram explaining aggregation processing of compressed variant information in one example of the embodiment;

FIG. 15 is a diagram exemplifying group statistical information in one example of the embodiment in a table format;

FIG. 16 is a diagram illustrating a first example of group dividing information in one example of the embodiment in a table format;

FIG. 17 is a diagram illustrating a second example of group dividing information in one example of the embodiment in a table format;

FIG. 18 is a flowchart explaining an operation example of variant information in one example of the embodiment;

FIG. 19 is a flowchart explaining compression processing of uncompressed variant information in one example of the embodiment;

FIG. 20 is a diagram explaining creation processing of compressed size information in one example of the embodiment;

FIG. 21 is a diagram explaining creation processing of combination compressed size information in one example of the embodiment;

FIG. 22 is a diagram explaining merge processing of combination compressed size information in one example of the embodiment;

FIG. 23 is a diagram exemplifying input data in compression processing of uncompressed variant information in one example of the embodiment;

FIG. 24 is a diagram exemplifying output data in compression processing of uncompressed variant information in one example of the embodiment;

FIG. 25 is a flowchart explaining details of compression processing of uncompressed variant information in one example of the embodiment;

FIG. 26 is a flowchart explaining creation processing of genome type data in one example of the embodiment;

FIG. 27 is a flowchart explaining aggregation processing of compressed variant information in one example of the embodiment;

FIG. 28 is a diagram exemplifying input data in creation processing of a temporary aggregation table in one example of the embodiment;

FIG. 29 is a diagram exemplifying output data in creation processing of a temporary aggregation table in one example of the embodiment;

FIG. 30 is a flowchart explaining creation processing of a temporary aggregation table in one example of the embodiment;

FIG. 31 is a diagram exemplifying input data in creation processing of a final aggregation table in one example of the embodiment;

FIG. 32 is a diagram exemplifying output data in creation processing of a final aggregation table in one example of the embodiment; and

FIG. 33 is a flowchart explaining creation processing of a final aggregation table in one example of the embodiment.

### DESCRIPTION OF EMBODIMENT

One embodiment will be described below with reference to the drawings. However, the embodiment to be represented below is merely exemplification and does not intend to exclude application of various modification examples and techniques that are not explicitly represented in the embodiment. For example, the present embodiment may be carried out with various modifications without departing from the gist thereof.

Each drawing is not made with intent that the embodiment includes only the constituent elements represented in the drawing, and the embodiment may include other functions and so forth.

In the following, each identical numeral represents similar part in the drawings and therefore description thereof is omitted.

### [A] Related Art

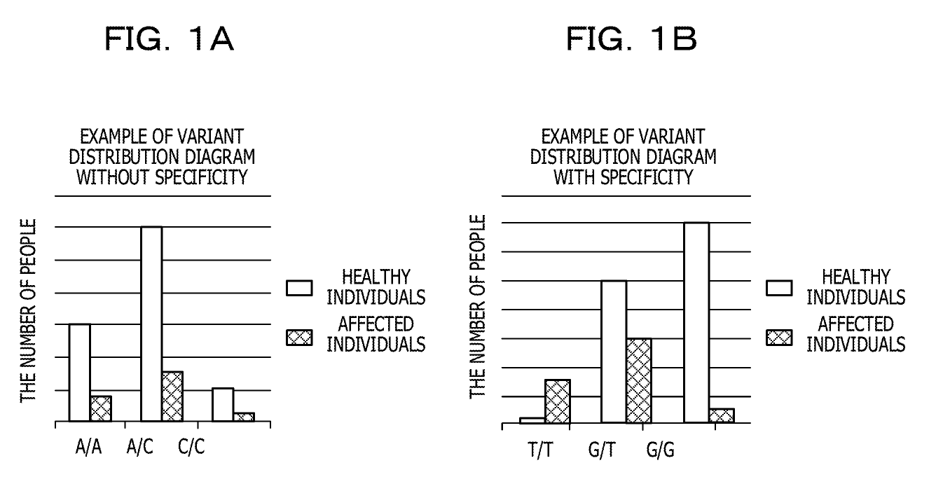

FIG. 1A is a graph illustrating one example of distribution of variant patterns in a variant without specificity. FIG. 1B is a graph illustrating one example of distribution of variant patterns in a variant with specificity.

Adenine (A), guanine (G), cytosine (C), and thymine (T) are included in the DNA sequence of the human. Each variant pattern in the DNA sequence is represented based on the combination of two of A, G, C, and T.

In FIG. 1A, the population distribution of each variant pattern in a certain variant having three kinds of variant patterns of A/A, A/C, and C/C is illustrated. In FIG. 1B, the population distribution of each variant pattern in a certain variant having three kinds of variant patterns of T/T, G/T, and G/G is illustrated.

In FIG. 1A and FIG. 1B, the “affected individuals” are persons who have a certain disease (for example, diabetes). The “healthy individuals” are persons who do not have the certain disease (for example, diabetes).

In the graph illustrated in FIG. 1A, the distributions of the healthy individuals and the affected individuals are similar in the three variant patterns. For example, the respective ratios between the variant patterns A/A, A/C, and C/C in the healthy individuals and the variant patterns A/A, A/C, and C/C in the affected individuals are substantially the same. On the other hand, in the graph illustrated in FIG. 1B, the distributions of the healthy individuals and the affected individuals are not similar in the three variant patterns. For example, the respective ratios between the variant patterns A/A, A/C, and C/C in the healthy individuals and the variant patterns A/A, A/C, and C/C in the affected individuals are not the same.

If the three variant patterns in a certain variant do not have a similar shape between the distributions of the healthy individuals and the affected individuals as illustrated in FIG. 1B, it is envisaged that this variant is a gene having a relation to the disease which these affected individuals have.

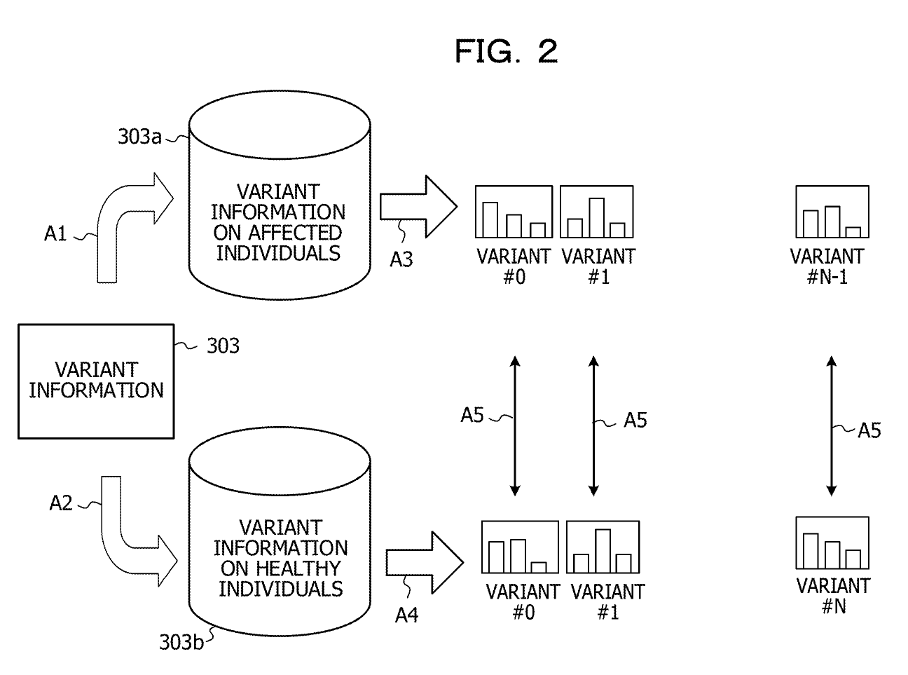

FIG. 2 is a block diagram illustrating an outline of aggregation processing of variant information.

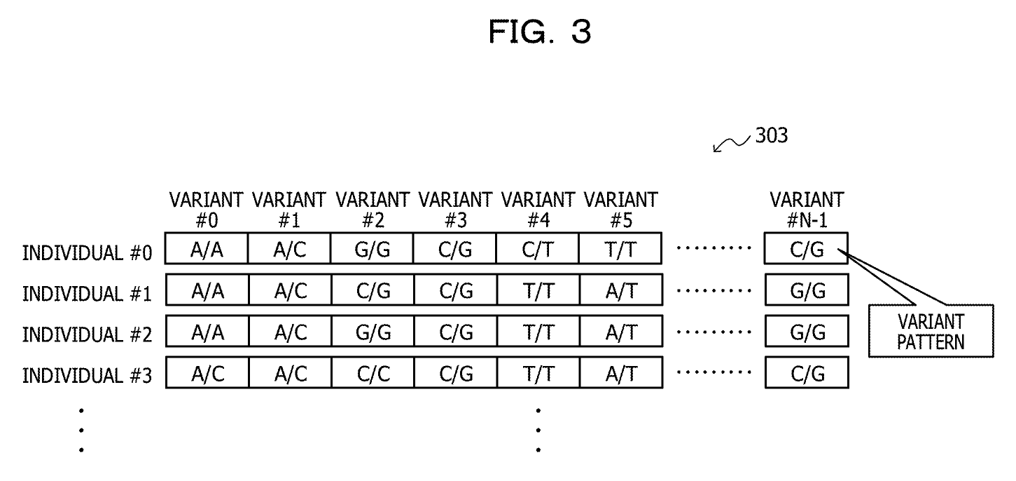

Variant information 303 illustrated in FIG. 2 is information that represents DNA sequences about plural individuals (individuals may be referred to as “humans”). Details of the variant information 303 will be described later by using FIG. 3.

The aggregation processing of the variant information 303 is executed in such a manner that each of variant information 303a on an affected individual group and variant information 303b on a healthy individual group is employed as a processing target. For this reason, as illustrated in FIG. 2, the variant information 303a on the affected individual group and the variant information 303b on the healthy individual group are each extracted from the variant information 303 (see symbols A1 and A2). Then, DNA sequences having N variants are each output from the variant information 303a on the affected individual group and the variant information 303b on the healthy individual group (see symbols A3 and A4).

Based on the variant information 303a on the affected individual group and the variant information 303b on the healthy individual group that are output, whether or not a significant difference exists in the appearance frequency of each variant pattern between the affected individual group and the healthy individual group is tested regarding each of individual variants by a statistical method such as a Chi-squared test (see symbols A5). The test represented by symbols A5 may be referred to as the “significant difference test.” The “appearance frequency of each variant pattern” may be referred to as the “distribution of the number of times of appearance of each variant pattern.”

FIG. 3 is a diagram illustrating one example of variant information.

The variant information 303 illustrated in FIG. 3 includes plural DNA sequences (DNA sequence may be referred to as “variant sequence” or simply as “sequence”). Plural variants are included in each DNA sequence. The contents of each variant are represented by the variant pattern. For example, the variant information 303 represents the variant pattern possessed by each of the plural variants included in the DNA sequence in each individual. The variant information 303 is difference information with respect to reference genome information. The reference genome information is information relating to the DNA sequence of a different race from the race of the analysis target of the DNA in some cases. For example, when pieces of variant information are collected with the Japanese people targeted, variant information on the human genome which the Japanese people have in common is extracted.

In the example illustrated in FIG. 3, the variant patterns of variants #0 to #N−1 in each of individuals #0, #1, #2, #3, . . . are represented. For example, in individual #0, the variant pattern of variant #0 is A/A. The variant pattern of variant #1 is A/C. The variant pattern of variant #2 is G/G.

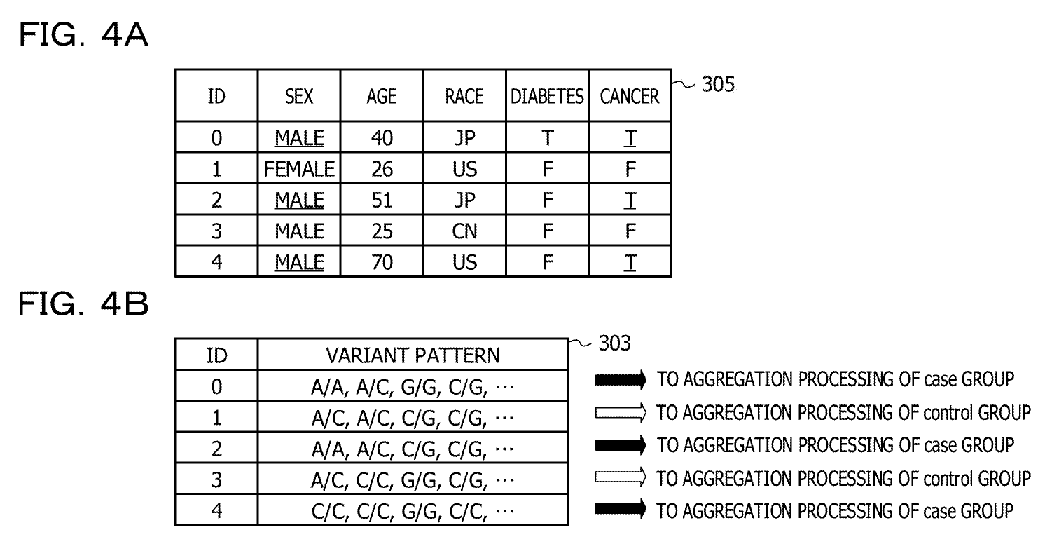

FIG. 4A is a diagram illustrating clinical information in a table format. FIG. 4B is a diagram illustrating variant information in a table format.

In clinical information 305 illustrated in FIG. 4A, attributes of each individual (individual may be referred to as “human”) are associated with information that represents whether or not disease exists.

In genome analysis, an individual is often extracted from the clinical information 305 in such a manner that characteristics such as whether or not disease exists, sex, age, and race are employed as a condition. In the extraction of the individual, the clinical information 305 and the variant information 303 are collated (for example, “JOIN”), and the group that matches the condition (this group may be referred to as “case group”) and the group that does not match the condition (this group may be referred to as “control group”) are extracted.

In the clinical information 305, “ID (identifier)” is information for uniquely identifying the individual. “Sex” represents the sex of the individual. “Age” represents the age of the individual and the unit of “age” is “years old.” “Race” represents the race of the individual. In the column of “race,” “JP” represents a Japanese person. “US” represents an American person. “CN” represents a Chinese person. “Diabetes” represents whether or not the individual is affected by diabetes. In the column of “diabetes,” “T” indicates that the individual is affected by diabetes, and “F” indicates that the individual is not affected by diabetes. “Cancer” represents whether or not the individual is affected by cancer. In the column of “cancer,” “T” indicates that the individual is affected by cancer, and “F” indicates that the individual is not affected by cancer.

In the clinical information 305, for example, the individuals whose “sex” is the male and who are affected by “cancer” are selected (see underlined parts in FIG. 4A). In FIG. 4A, “ID” of the individuals whose “sex” is the male and who are affected by “cancer” is “0”“2,” and “4.”

In the variant information 303 illustrated in FIG. 4B, the ID of each individual is associated with the variant patterns.

In the variant information 303, “ID” is information for uniquely identifying the individual and corresponds to “ID” of the clinical information 305. “Variant pattern” represents the patterns of variants included in the DNA sequence of each individual.

In the example illustrated in FIG. 4B, the variant patterns whose “ID” is “0,”“2,” and “4” selected in the above description of FIG. 4A are extracted for aggregation processing of the case group. The variant patterns whose “ID” is “1” and “3,” which are not selected in the above description of FIG. 4A, are extracted for aggregation processing of the control group.

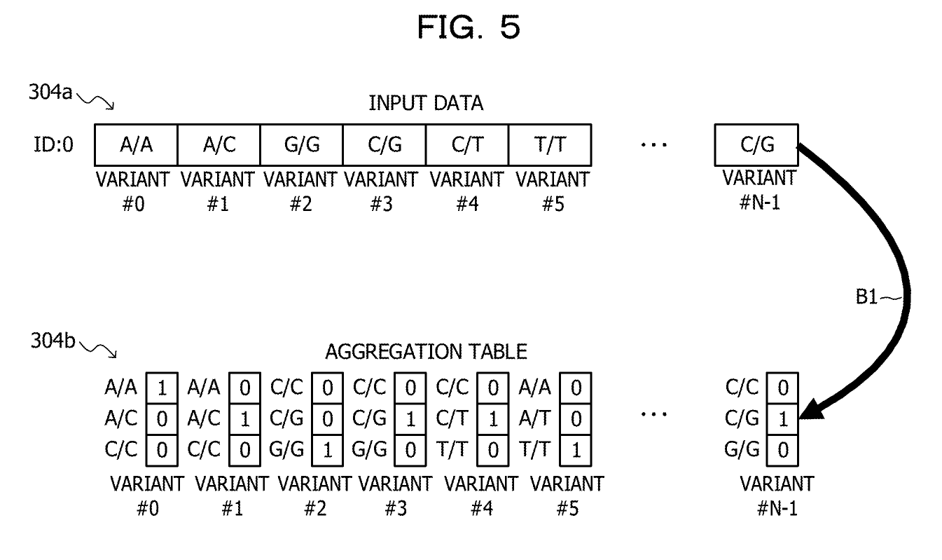

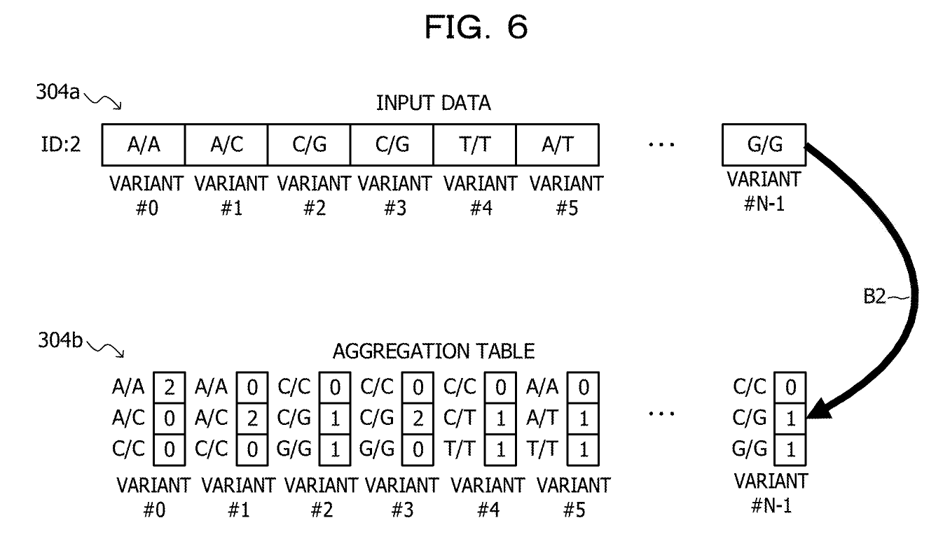

FIG. 5 and FIG. 6 are diagrams explaining aggregation processing of variant sequences.

In the aggregation processing of variant sequences, the variant pattern included in each variant is counted regarding each “ID” of the case group and the control group extracted in FIG. 4B.

In FIG. 5, the variant patterns of the individual whose “ID” is “0” extracted for the aggregation processing of the case group are employed as input data 304a and the variant pattern of each variant is counted by an aggregation table 304b (see symbol B1).

In the aggregation table 304b, corresponding to the variant patterns of the input data 304a, for example, the counts of the variant pattern “A/A” of variant #0, the variant pattern “A/C” of variant #1, and the variant pattern “G/G” of variant #2 are incremented from 0 to 1. The counts in variants #3 to #N−1 are also incremented corresponding to the variant patterns of the input data 304a similarly.

Next, in FIG. 6, the variant patterns of the individual whose “ID” is “2” extracted for the aggregation processing of the case group are employed as the input data 304a and the variant pattern of each variant is counted by the aggregation table 304b (see symbol B2).

In the aggregation table 304b, corresponding to the variant patterns of the input data 304a, for example, the counts of the variant pattern “A/A” of variant #0 and the variant pattern “A/C” of variant #1 are incremented from 1 to 2. For example, the count of the variant pattern “C/G” of variant #2 is incremented from 0 to 1. Moreover, the counts in variants #3 to #N−1 are also incremented corresponding to the variant patterns of the input data 304a similarly.

By repeating the processing represented by the processing B1 of FIG. 5 and the processing B2 of FIG. 6 the same number of times as the number of individuals extracted for the aggregation processing of the case group in FIG. 4B, the aggregation processing of the case group is completed. The aggregation processing of the control group is also executed similarly to the aggregation processing of the case group.

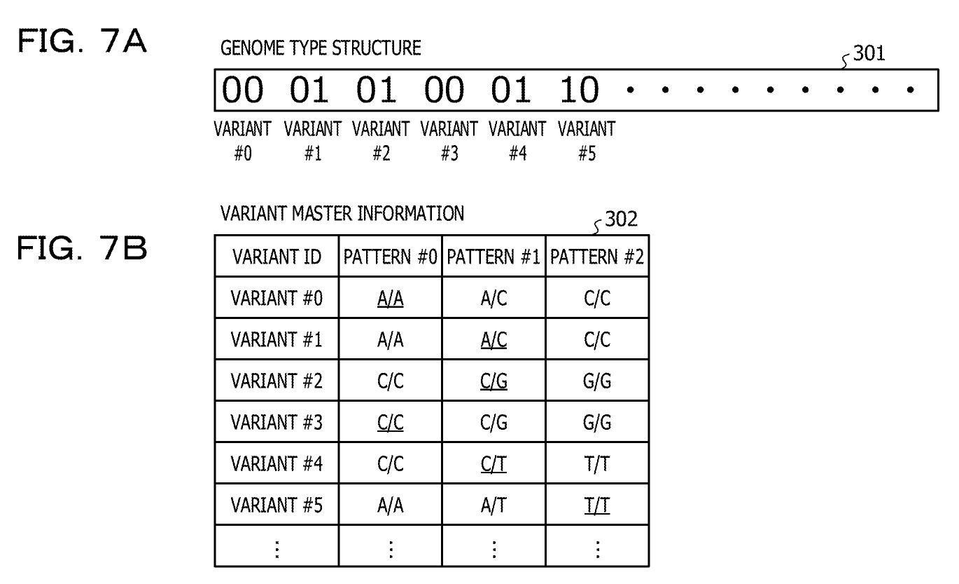

FIG. 7A is a diagram exemplifying a genome type structure. FIG. 7B is a diagram illustrating variant master information in a table format.

A genome type structure 301 illustrated in FIG. 7A is information that represents each of the variant patterns of the respective variants in a certain variant sequence by 2 bits.

Variant master information 302 illustrated in FIG. 7B is information that manages which position in the genome type structure 301 each variant corresponds to and which variant pattern each variant has.

Many of the respective variants included in the DNA sequence are represented by any of three variant patterns (for example, A/A, A/C, and C/C for variant #0 in FIG. 7B). Thus, a storage area of 2 bits is allocated to each variant. This may store the three variant patterns in the storage area of 2 bits. At most four variant patterns may be stored in the storage area of 2 bits.

In the example illustrated in FIG. 7B, in variant #0, pattern #0 is A/A and pattern #1 is A/C and pattern #2 is C/C. In each variant, pattern #0 is represented by “00” and pattern #1 is represented by “01”and pattern #2 is represented by “10.”

If the variant patterns of variants #0 to #5 are “A/A, A/C, C/G, C/C, C/T, T/T” as represented by underlined parts in FIG. 7B, the genome type structure 301 becomes “000101000110” as illustrated in FIG. 7A.

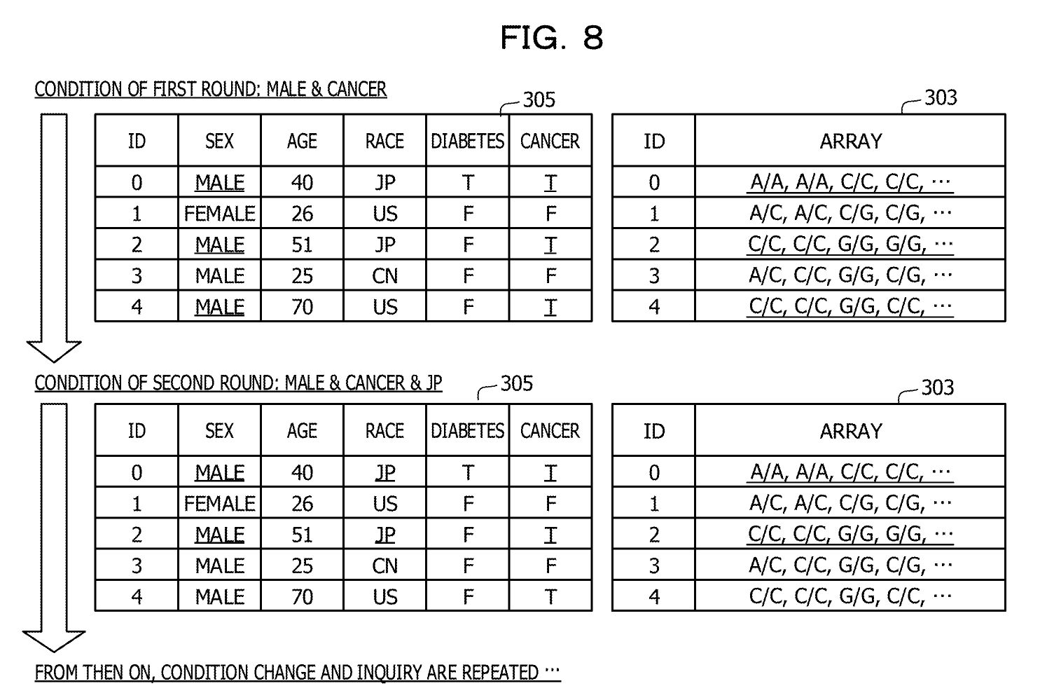

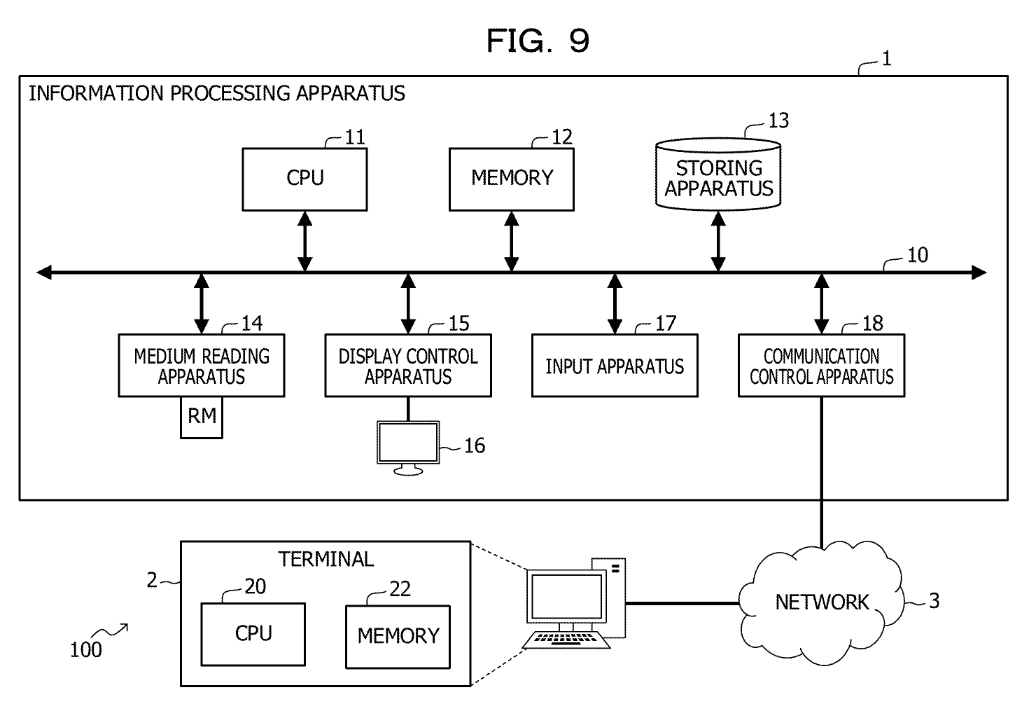

FIG. 8 is a diagram explaining retrieval processing (retrieval processing may be referred to as “analysis processing”) of variant information. The retrieval processing may be executed by an inquiry from a terminal 2 to be described later by using FIG. 9 to an information processing apparatus 1.

In the example illustrated in FIG. 8, as the condition of the first round of retrieval, the individuals whose “sex” is the male and who are affected by “cancer” are retrieved in the clinical information 305 (see underlined parts in “clinical information 305” with symbol C1). Due to this, in the variant information 303, the variant patterns whose “ID” is 0, 2, and 4 are extracted as the inquiry result (see underlined parts in “variant information 303” with symbol C1).

Next, as the condition of the second round of retrieval, the individuals whose “sex” is the male and who are affected by “cancer” and whose “race” is Japanese are detected in the clinical information 305 (see underlined parts in “clinical information 305” with symbol C2). Due to this, in the variant information 303, the variant patterns whose “ID” is 0 and 2 are extracted as the inquiry result (see underlined parts in “variant information 303” with symbol C2).

From then on, with viewing of the inquiry result, interactive processing of changing the retrieval condition and making an inquiry again is repeatedly executed.

Approximately 20 million variants are included in the variant information on the human genome. Because information of 2 bits is held per one variant, the data amount of variant information regarding 100 thousand people is approximately 500 GB. If the data capacity of a primary storing apparatus of a computer used for retrieval and analysis of the variant information on the human genome is not lower than the data amount of the variant information, access to a secondary storing apparatus occurs in processing of retrieval and analysis. This possibly lowers the processing speed of retrieval and analysis of the variant information on the human genome.

Thus, it is envisaged that the variant information 303 is compressed by using an existing data compression technique and the compressed data is used while being loaded in a memory. However, also in this case, possibly the processing speed becomes low due to the loading of the compressed data in the memory.

### [B] One Example of Embodiment

In the DNA sequence, when group dividing is carried out based on the race, sex, age, and so forth, there are a large number of variants having the same variant pattern among all members (members may be referred to as “individuals”) in the group. For example, in the DNA sequence of the Japanese people, 800 thousand variants in 3 million variants in chromosome 1 have the same variant pattern.

Thus, in one example of the embodiment, if the variant patterns possessed by the variants that correspond have the same value among plural DNA sequences, these variant patterns are not stored in a memory. This reduces the amount of data stored in the memory and improves the analysis speed of the DNA sequences.

### [B-1] Hardware Configuration Example

FIG. 9 is a block diagram illustrating a hardware configuration of an information processing system in one example of the embodiment.

An information processing system 100 illustrated in FIG. 9 includes the information processing apparatus 1 and the terminal 2. The information processing apparatus 1 and the terminal 2 may be coupled communicably with each other through a network 3.

The terminal 2 is a computer used by a user. The user may execute analysis processing on variant information compressed by compression processing in the one example of the embodiment by using this terminal 2. Exemplarily, the terminal 2 includes a central processing unit (CPU) 20 and a memory 22. Similarly to the information processing apparatus 1, the terminal 2 may include a storing apparatus 13, a medium reading apparatus 14, a display control apparatus 15, a display apparatus 16, an input apparatus 17, and a communication control apparatus 18 to be each described later.

The memory 22 is one example of a storing unit and exemplarily is a storing apparatus including at least one of a read only memory (ROM) and a random access memory (RAM). A program such as a basic input/output system (BIOS) may be written to the ROM of the memory 22. A software program of the memory 22 may be read into the CPU 20 as appropriate to be executed. The RAM of the memory 22 may be used as a primary recording memory or working memory. The memory 22 may store a genome type structure 201, variant master information 202, original data variant information 203, uncompressed variant information 204, clinical information 205, compressed variant information 206, a temporary aggregation table 207, and a final aggregation table 208 to be described later. The memory 22 may store group statistical information 209, NULL variant aggregation information 209a, compressed size information 209b, group dividing information 210, combination NULL variant aggregation information 210a, and combination compressed size information 210b to be described later. Furthermore, the memory 22 may store ranking information 211, NULL variant structures 212a and 212b, a group ID correspondence array 213, and a combination 214 to be described later.

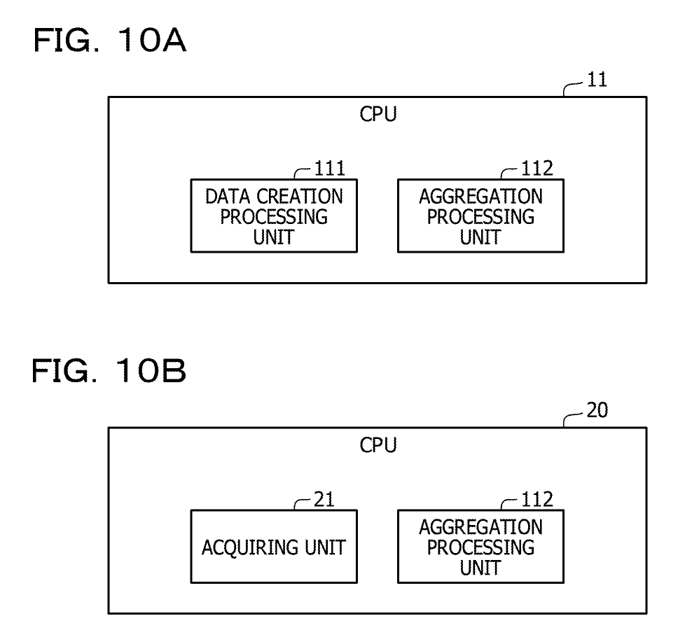

The CPU 20 is a processing apparatus that carries out various kinds of control and arithmetic operation and implements various functions by executing an operating system (OS) and programs stored in the memory 22. Functions of the CPU 20 will be described later by using FIG. 10B.

Exemplarily, the information processing apparatus 1 includes a CPU 11, a memory 12, the storing apparatus 13, the medium reading apparatus 14, the display control apparatus 15, the display apparatus 16, the input apparatus 17, and the communication control apparatus 18. The CPU 11, the memory 12, the storing apparatus 13, the medium reading apparatus 14, the display control apparatus 15, the input apparatus 17, and the communication control apparatus 18 are coupled communicably with each other through a bus line 10.

Exemplarily, the storing apparatus 13 is an apparatus that stores data in a readable-writable manner and hard disk drive (HDD), solid state drive (SSD), and storage class memory (SCM) may be used, for example. The storing apparatus 13 may store the genome type structure 201, the variant master information 202, the original data variant information 203, the uncompressed variant information 204, the clinical information 205, the compressed variant information 206, the temporary aggregation table 207, and the final aggregation table 208 to be described later. The storing apparatus 13 may store the group statistical information 209, the NULL variant aggregation information 209a, the compressed size information 209b, the group dividing information 210, the combination NULL variant aggregation information 210a, and the combination compressed size information 210b to be described later. Furthermore, the storing apparatus 13 may store the ranking information 211, the NULL variant structures 212a and 212b, the group ID correspondence array 213, and the combination 214 to be described later.

The medium reading apparatus 14 is configured in such a manner that a recording medium RM may be mounted thereto. The medium reading apparatus 14 is configured to be capable of reading information recorded on the recording medium RM in the state in which the recording medium RM is mounted. In the present example, the recording medium RM has portability. The recording medium RM is a computer-readable recording medium and is a flexible disc, compact disc (CD), digital versatile disc (DVD), Blu-ray Disc, magnetic disc, optical disc, magneto-optical disc, semiconductor memory, or the like, for example. The CD may be a CD-read only memory (ROM), CD-recordable (R), CD-rewritable (RW), or the like. The DVD may be a DVD-ROM, DVD-random access memory (RAM), DVD-R, DVD+R, DVD-RW, DVD+RW, high-definition (HD) DVD, or the like.

The display control apparatus 15 is communicably coupled to the display apparatus 16 and controls screen displaying of the display apparatus 16.

The display apparatus 16 is a liquid crystal display, cathode ray tube (CRT), electronic paper display, or the like and displays various kinds of information for an operator or the like.

The input apparatus 17 is a mouse, trackball, or keyboard, for example, and an operator carries out various kinds of input operation through this input apparatus 17.

The display apparatus 16 and the input apparatus 17 may be combined and may be a touch panel, for example.

The communication control apparatus 18 controls communication between the information processing apparatus 1 and the network 3. The communication control apparatus 18 may control communication between the information processing apparatus 1 and another computer such as the terminal 2 through the network 3.

The memory 12 is one example of a storing unit and exemplarily is a storing apparatus including at least one of ROM and RAM. A program such as a BIOS may be written to the ROM of the memory 12. A software program of the memory 12 may be read into the CPU 11 as appropriate to be executed. The RAM of the memory 12 may be used as a primary recording memory or working memory. The memory 12 may store the genome type structure 201, the variant master information 202, the original data variant information 203, the uncompressed variant information 204, the clinical information 205, the compressed variant information 206, the temporary aggregation table 207, and the final aggregation table 208 to be described later. The memory 12 may store the group statistical information 209, the NULL variant aggregation information 209a, the compressed size information 209b, the group dividing information 210, the combination NULL variant aggregation information 210a, and the combination compressed size information 210b to be described later. Furthermore, the memory 12 may store the ranking information 211, the NULL variant structures 212a and 212b, the group ID correspondence array 213, and the combination 214 to be described later.

The CPU 11 is a processing apparatus that carries out various kinds of control and arithmetic operation. The CPU 11 implements various functions by executing an OS and programs stored in the memory 12.

FIG. 10A is a block diagram illustrating a functional configuration of an information processing apparatus in one example of the embodiment.

As illustrated in FIG. 10A, the CPU 11 functions as a data creation processing unit 111 and an aggregation processing unit 112.

A program for implementing functions as these data creation processing unit 111 and aggregation processing unit 112 is provided in the form of being recorded on the above-described recording medium RM, for example. Furthermore, a computer reads the program from the recording medium RM through the medium reading apparatus 14 and transfers the program to an internal storing apparatus or an external storing apparatus to store and use it. Alternatively, the program may be recorded on a storing apparatus (recording medium) such as a magnetic disc, optical disc, or magneto-optical disc and be provided from the storing apparatus to the computer through a communication path

When the functions as the data creation processing unit 111 and the aggregation processing unit 112 are implemented, the program stored in the internal storing apparatus (in the present embodiment, memory 12) is executed by a microprocessor (in the present embodiment, CPU 11) of the computer. At this time, the computer may read and execute the program recorded on the recording medium RM.

The information processing apparatus 1 may include any one of MPU, DSP, ASIC, PLD, and FPGA instead of the CPU 11. The information processing apparatus 1 may combine and include two or more kinds of CPU, MPU, DSP, ASIC, PLD, and FPGA. The MPU is an abbreviation for micro processing unit. The DSP is an abbreviation for digital signal processor. The ASIC is an abbreviation for application specific integrated circuit. The PLD is an abbreviation for programmable logic device. The FPGA is an abbreviation for field programmable gate array.

The data creation processing unit 111 causes the memory 12 to store plural variant patterns included in each of plural DNA sequences. If variant patterns that correspond have the same value among plural sequences, the data creation processing unit 111 excludes these variant patterns from the storing target of the memory 12.

For example, the data creation processing unit 111 is one example of a processing unit and executes processing of exclusion of variant patterns having the same value from the storing target if the variant patterns at the same variant position have the same value among plural sequences each including plural variant patterns. The data creation processing unit 111 causes the memory 12 to store the plural sequences for which the processing of exclusion has been executed.

The data creation processing unit 111 may restore the sequences before the execution of the processing of exclusion by inserting the variant patterns deemed as the target of the processing of exclusion into the sequences for which the processing of exclusion has been executed based on the group dividing information 210 to be described later. The group dividing information 210 may be referred to as information that represents the position of the variant patterns deemed as the target of the processing of exclusion.

The aggregation processing unit 112 carries out analysis of DNA sequences based on the variant patterns stored in the memory 12 by the data creation processing unit 111. If analysis of DNA sequences is carried out in the terminal 2 illustrated in FIG. 9, functions as the aggregation processing unit 112 may be included in the terminal 2.

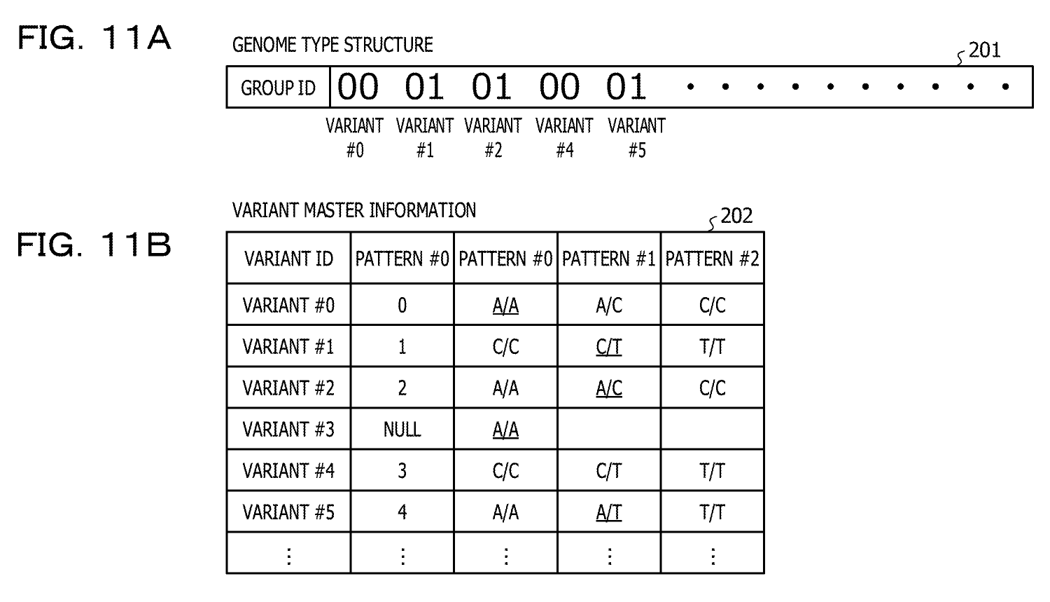

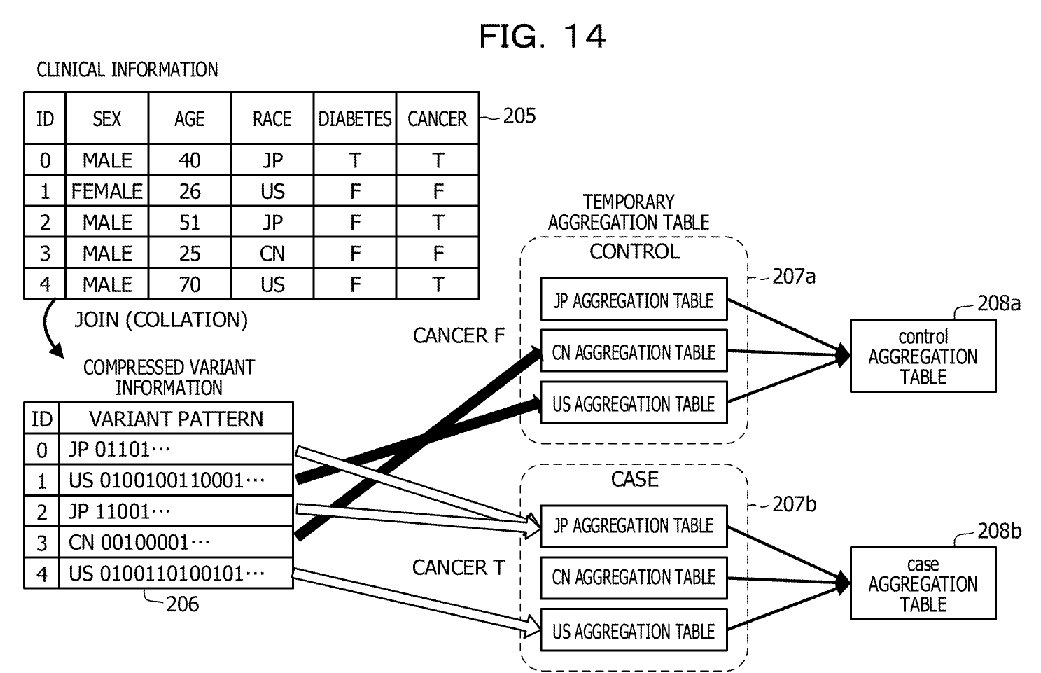

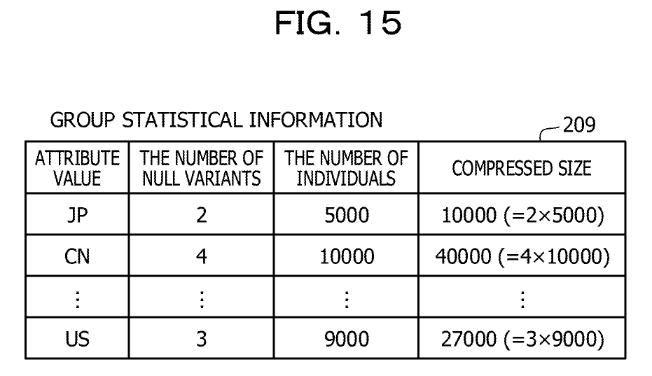

Details of the data creation processing unit 111 will be described later by using FIG. 11 to FIG. 13, FIG. 15 to FIG. 17, and so forth. Details of the aggregation processing unit 112 will be described later by using FIG. 14 and so forth.

FIG. 10B is a block diagram illustrating a functional configuration of a terminal in one example of the embodiment.

As illustrated in FIG. 10B, the CPU 20 functions as an acquiring unit 21 and the aggregation processing unit 112.

A program for implementing functions as these acquiring unit 21 and aggregation processing unit 112 is provided in the form of being recorded on a recording medium, for example. Furthermore, a computer reads the program from the recording medium through a medium reading apparatus (not illustrated) and transfers the program to an internal storing apparatus or an external storing apparatus to store and use it. Alternatively, the program may be recorded on a storing apparatus (recording medium) such as a magnetic disc, optical disc, or magneto-optical disc and be provided from the storing apparatus to the computer through a communication path.

When the functions as the acquiring unit 21 and the aggregation processing unit 112 are implemented, the program stored in the internal storing apparatus (in the present embodiment, memory 22) is executed by a microprocessor (in the present embodiment, CPU 20) of the computer. At this time, the computer may read and execute the program recorded on the recording medium.

The terminal 2 may include any one of MPU, DSP, ASIC, PLD, and FPGA instead of the CPU 20. The terminal 2 may combine and include two or more kinds of CPU, MPU, DSP, ASIC, PLD, and FPGA.

The acquiring unit 21 acquires various kinds of data from the information processing apparatus 1 through the network 3 (see FIG. 9), for example, and causes the memory 22 to store the acquired data. The genome type structure 201, the variant master information 202, the original data variant information 203, the uncompressed variant information 204, the clinical information 205, the compressed variant information 206, the temporary aggregation table 207, and the final aggregation table 208 to be described later may be included in the various kinds of data. The group statistical information 209, the NULL variant aggregation information 209a, the compressed size information 209b, the group dividing information 210, the combination NULL variant aggregation information 210a, and the combination compressed size information 210b to be described later may be included in the various kinds of data. Furthermore, the ranking information 211, the NULL variant structures 212a and 212b, the group ID correspondence array 213, and the combination 214 to be described later may be included in the various kinds of data.

The acquiring unit 21 may specify a group used for compression of variant patterns by the information processing apparatus 1 and acquire the variant patterns compressed by the specified group from the information processing apparatus 1. The acquiring unit 21 may cause the memory 22 to store the acquired variant patterns.

For example, as described above by using FIG. 8, the acquiring unit 21 specifies the retrieval condition based on a group of the sex, race, and so forth and makes an inquiry to the information processing apparatus 1. Then, the acquiring unit 21 acquires variant patterns compressed based on the specified retrieval condition from the information processing apparatus 1.

The acquiring unit 21 may restore the sequences before execution of processing of exclusion by inserting variant patterns deemed as the target of the processing of exclusion into the sequences for which the processing of exclusion has been executed based on the group dividing information 210 to be described later. The group dividing information 210 may be referred to as information that represents the position of the variant patterns deemed as the target of the processing of exclusion.

FIG. 11A is a diagram illustrating a genome type structure. FIG. 11B is a diagram illustrating variant master information in a table format.

The genome type structure 201 illustrated in FIG. 11A is information that represents each of the variant patterns of the respective variants in a certain variant sequence by 2 bits. In the beginning region of the genome type structure 201, “group ID” that is an identifier for identifying the group to which the relevant variant sequence belongs is added.

The variant master information 202 illustrated in FIG. 11B is information that manages which position in the genome type structure 201 each variant corresponds to and which variant pattern each variant has. The variant master information 202 has a column of “genome type position” and NULL is set for the variants whose variant pattern is limited to one kind. In addition, the variant master information 202 has information regarding which position in the genome type structure 201 the variants other than the variants whose variant pattern is limited to one kind correspond to.

Many of the respective variants included in the DNA sequence are represented by any of three variant patterns (for example, A/A, A/C, and C/C for variant #0 in FIG. 11B). Thus, a storage area of 2 bits is allocated to each variant. This may store the three variant patterns in the storage area of 2 bits. At most four variant patterns may be stored in the storage area of 2 bits.

In the example illustrated in FIG. 11B, in variant #0, pattern #0 is A/A and pattern #1 is A/C and pattern #2 is C/C. For each variant, the variant patterns of patterns #0, #1, and #2 in FIG. 11B are converted to “00,”“01,” and “10,” respectively, and are stored in the genome type structure 201 in FIG. 11A.

In the example illustrated in FIG. 11B, the variant pattern in variant #3 is limited to A/A of pattern #0. The data creation processing unit 111 sets “genome type position” of the variants whose variant pattern is limited to one kind to NULL. Meanwhile, the data creation processing unit 111 registers values of 0, 1, 2, 3, 4, . . . sequentially from the variant with the smallest “variant ID” in “genome type position” of the variants other than the variants whose variant pattern is limited to one kind.

If the variant patterns of variants #0 to #5 are “A/A, C/T, A/C, A/A, C/C, A/T” as represented by underlined parts in FIG. 11B, the genome type structure 201 becomes “0001010001” as illustrated in FIG. 11A. As illustrated in FIG. 11A, the data creation processing unit 111 does not register the variant pattern of variant #3, whose variant pattern is limited to one kind, in the genome type structure 201. Meanwhile, the data creation processing unit 111 registers, in the genome type structure 201, the variant patterns of variants #0 to #2, #4, #5, . . . other than the variants whose variant pattern is limited to one kind.

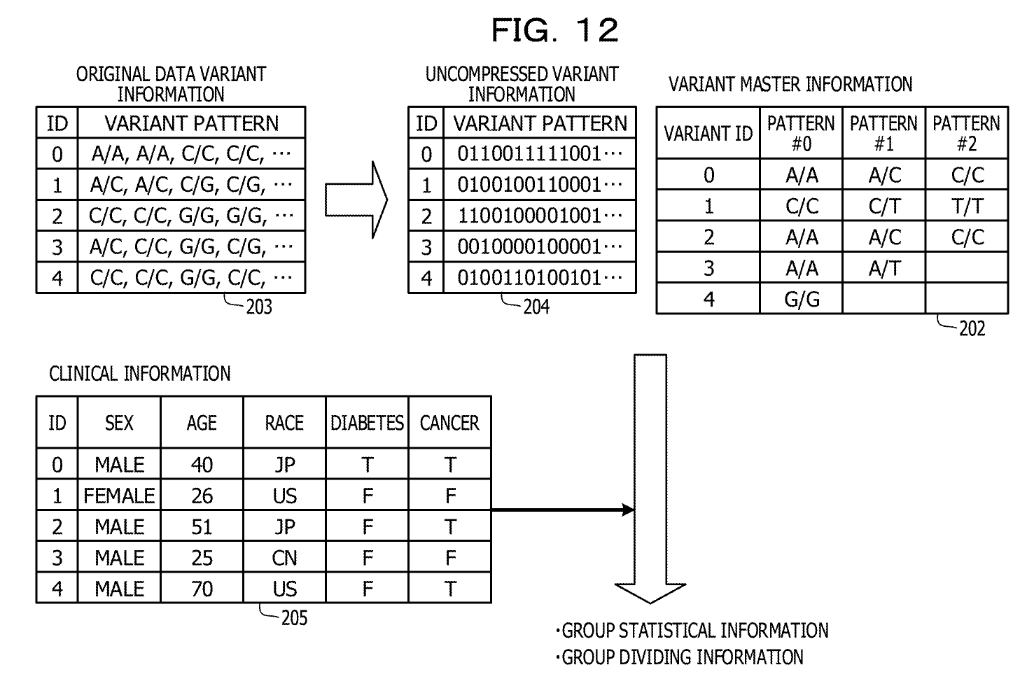

FIG. 12 is a diagram explaining creation processing of group statistical information and group dividing information in one example of the embodiment.

The data creation processing unit 111 creates the uncompressed variant information 204 and the variant master information 202 based on the original data variant information 203.

The original data variant information 203 is information that represents, by AGCT, the variant pattern possessed by each of the plural variants included in the DNA sequence in each individual.

The uncompressed variant information 204 is information that represents, by data of 2 bits, the variant pattern possessed by each of the plural variants included in the DNA sequence in each individual. The conversion from the original data variant information 203 to the uncompressed variant information 204 is carried out by the method explained by using FIG. 11.

The data creation processing unit 111 creates the group statistical information 209 and the group dividing information 210 based on the clinical information 205, the created uncompressed variant information 204, and the variant master information 202.

The clinical information 205 is information that associates attributes of each individual (individual may be referred to as “human”) with information that represents whether or not disease exists.

In the clinical information 205, “ID” is information for uniquely identifying the individual. “Sex” represents the sex of the individual. “Age” represents the age of the individual and the unit of “age” is “years old.” “Race” represents the race of the individual. In the column of “race,”“JP” represents a Japanese person. “US” represents an American person. “CN” represents a Chinese person. “Diabetes” represents whether or not the individual is affected by diabetes. In the column of “diabetes,” “T” indicates that the individual is affected by diabetes, and “F” indicates that the individual is not affected by diabetes. “Cancer” represents whether or not the individual is affected by cancer. In the column of “cancer,” “T” indicates that the individual is affected by cancer, and “F” indicates that the individual is not affected by cancer. The race may be the nationality, hometown, or the like.

The group statistical information 209 is information that represents a compressed size caused by keeping the memory 12 from storing the variants whose variant pattern is one kind in the case in which DNA sequences are extracted regarding each of the attributes such as “sex” and “race” in the clinical information 205. Details of the group statistical information 209 will be described later by using FIG. 15. In the present specification, the “compressed size” represents the size by which the data amount is reduced by compression processing of data.

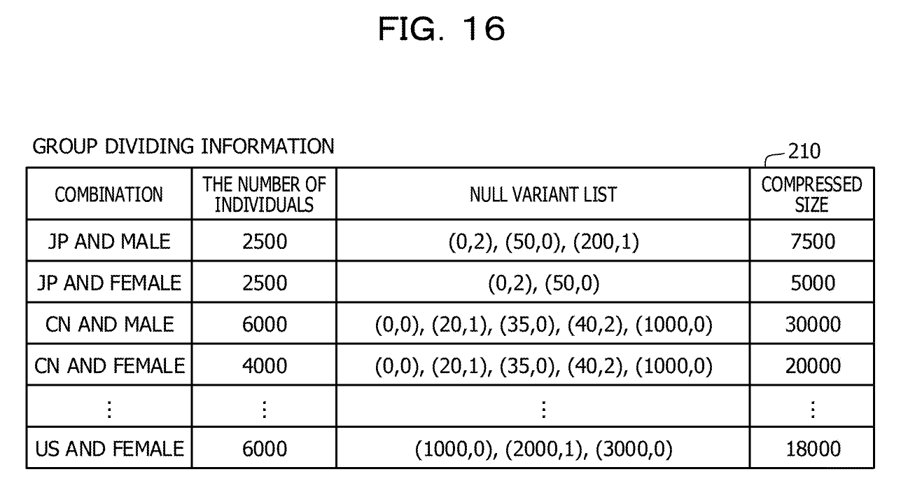

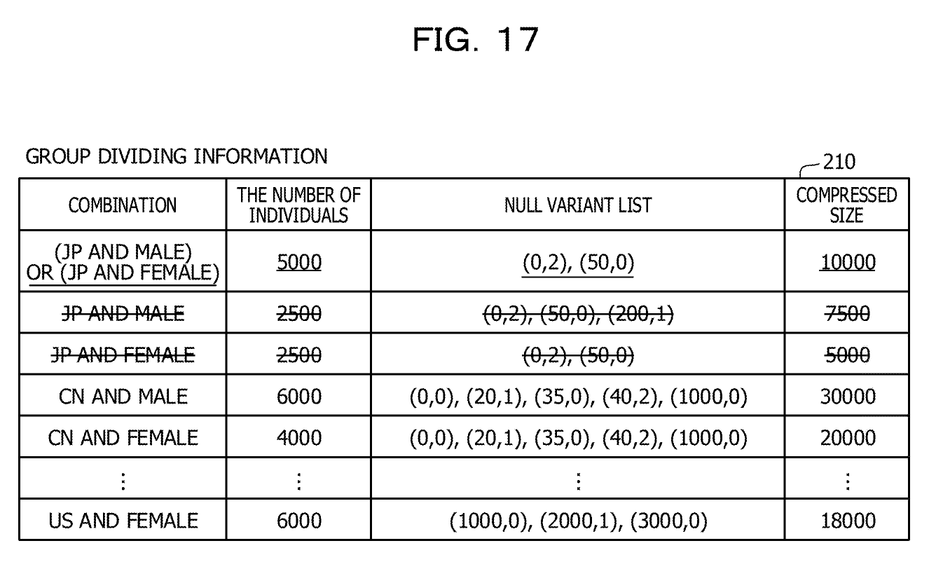

The group dividing information 210 is information that represents a compressed size caused by keeping the memory 12 from storing the variants whose variant pattern is one kind in the case in which DNA sequences are extracted regarding combinations of plural attributes. Details of the group dividing information 210 will be described later by using FIG. 16 and FIG. 17.

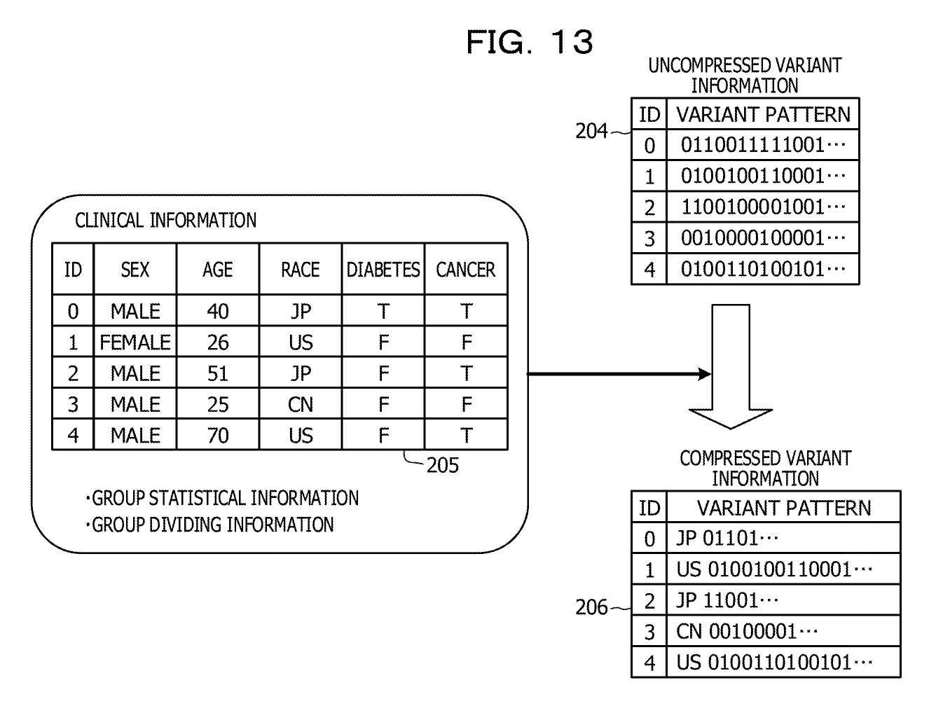

FIG. 13 is a diagram explaining compression processing of uncompressed variant information in one example of the embodiment.

The data creation processing unit 111 creates the compressed variant information 206 based on the clinical information 205 and the created group statistical information 209 and group dividing information 210.

The compressed variant information 206 is information that represents, by data of 2 bits, the variant pattern possessed by each of the plural variants included in the DNA sequence in each individual. In “variant pattern” of the compressed variant information 206, the variant patterns registered in “NULL variant list” in the group dividing information 210 to be described later are deleted. Due to this, at least partial variant sequences in the plural variant sequences registered in the compressed variant information 206 become shorter than variant sequences of the uncompressed variant information 204.

In the beginning region of the compressed variant information 206, “group ID” that is an identifier for identifying the group to which the relevant variant sequence belongs is added. In the example illustrated in FIG. 13, JP, US, and CN representing the race are associated with “group ID” of the compressed variant information 206.

FIG. 14 is a diagram explaining aggregation processing of compressed variant information in one example of the embodiment.

The aggregation processing unit 112 registers the variant patterns of the compressed variant information 206 in a temporary aggregation table 207a of the control group and a temporary aggregation table 207b of the case group by carrying out collation of the clinical information 205 (collation may be referred to as “JOIN”). In the example illustrated in FIG. 14, the variant patterns of the individuals who are not affected by cancer in the variant patterns of the compressed variant information 206 divided into groups regarding each race are registered in the temporary aggregation table 207a of the control group. The variant patterns of the individuals who are affected by cancer in the variant patterns of the compressed variant information 206 divided into groups regarding each race are registered in the temporary aggregation table 207b of the case group.

In the example illustrated in FIG. 14, JP aggregation table, CN aggregation table, and US aggregation table are included in each of the temporary aggregation table 207a of the control group and the temporary aggregation table 207b of the case group.

At ID=0 of the compressed variant information 206, JP is added to the variant patterns as the group ID and the individual is affected by cancer when the clinical information 205 is collated. Therefore, the variant patterns of ID=0 are registered in the JP aggregation table of the case group. At ID=1 of the compressed variant information 206, US is added to the variant patterns as the group ID and the individual is not affected by cancer when the clinical information 205 is collated. Therefore, the variant patterns of ID=1 are registered in the US aggregation table of the control group. At ID=2 of the compressed variant information 206, JP is added to the variant patterns as the group ID and the individual is affected by cancer when the clinical information 205 is collated. Therefore, the variant patterns of ID=2 are registered in the JP aggregation table of the case group. At ID=3 of the compressed variant information 206, CN is added to the variant patterns as the group ID and the individual is not affected by cancer when the clinical information 205 is collated. Therefore, the variant patterns of ID=3 are registered in the CN aggregation table of the control group. At ID=4 of the compressed variant information 206, US is added to the variant patterns as the group ID and the individual is affected by cancer when the clinical information 205 is collated. Therefore, the variant patterns of ID=4 are registered in the US aggregation table of the case group.

The aggregation processing unit 112 combines the JP aggregation table, the CN aggregation table, and the US aggregation table of the control group to create a control aggregation table 208a. The aggregation processing unit 112 combines the JP aggregation table, the CN aggregation table, and the US aggregation table of the case group to create a case aggregation table 208b.

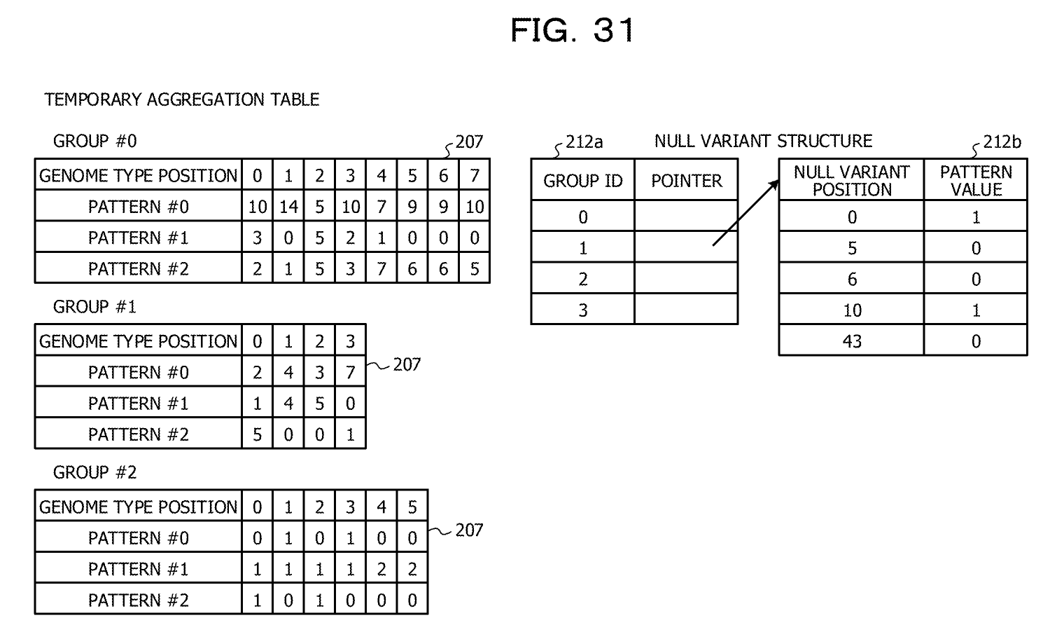

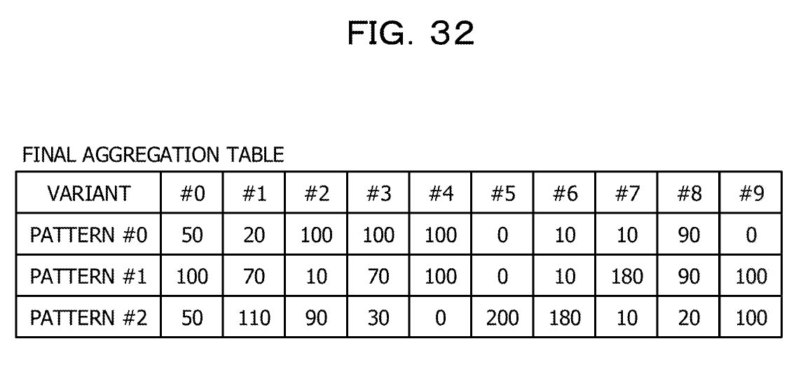

Details of the temporary aggregation table 207 (for example, “temporary aggregation table 207a of the control group” and “temporary aggregation table 207b of the case group”) will be described later by using FIG. 31 and so forth. The final aggregation table 208 (for example, “control aggregation table 208a” and “case aggregation table 208b”) will be described later by using FIG. 32 and so forth.

The data creation processing unit 111 may select combinations with which the compression rate of the data size becomes high from combinations of attribute conditions of the clinical information 205 and carry out grouping. The data creation processing unit 111 may set the upper limit of the number of grouped combinations to N_{G} and select up to the upper-limit number N_{G} of combinations.

FIG. 15 is a diagram exemplifying group statistical information in one example of the embodiment in a table format. The group statistical information 209 exemplified in FIG. 15 represents the compressed size about the variant patterns in each race.

The data creation processing unit 111 creates the group statistical information 209 exemplified in FIG. 15. The data creation processing unit 111 may create the group statistical information 209 about an attribute other than “race,” such as “sex” or “age.”

In the column of “attribute value,” members of any attribute in the plural attributes included in the clinical information 205 are registered. In the example illustrated in FIG. 15, JP, CN, and US are registered in the column of “attribute value.”

“The number of NULL variants” represents the number of variants that are identical among all individuals having the attribute value (these variants may be referred to as “NULL variants”).

“The number of individuals” represents the number of individuals having the attribute value.

“Compressed size” represents the data size compressed by the NULL variants and is calculated based on the product of “the number of NULL variants” and “the number of individuals.” Through summation of “compressed size” of the respective attribute values, the total of the compressed size when grouping is carried out based on this attribute is calculated. In the example illustrated in FIG. 15, the sum of the compressed size when grouping is carried out based on the attribute “race” is calculated.

FIG. 16 is a diagram illustrating a first example of group dividing information in one example of the embodiment in a table format.

The group dividing information 210 illustrated in FIG. 16 is information that represents the positions of the variant patterns deemed as the target of processing of exclusion. The data creation processing unit 111 creates the group dividing information 210 exemplified in FIG. 16 based on the created group statistical information 209.

“Combination” represents combinations of plural attribute values. In the example illustrated in FIG. 16, “JP and Male” represents individuals whose race is Japanese and whose sex is the male, for example.

“The number of individuals” represents the number of individuals having the attribute values.

“NULL variant list” represents the position (for example, “genome type position”) of the NULL variant and the value of the variant pattern of this NULL variant. In FIG. 16, “NULL variant list” is represented in a format of (position of NULL variant, value of variant pattern). For example, (0, 2) indicates that variant #0 is a NULL variant and the variant pattern of variant #0 is pattern #2.

“Compressed size” represents the data size compressed due to the NULL variants and is calculated based on the product of the number of NULL variants included in “NULL variant list” and “the number of individuals.”

FIG. 17 is a diagram illustrating a second example of group dividing information in one example of the embodiment in a table format.

The data creation processing unit 111 may register combinations of attributes with a large compressed size in the group dividing information 210 sequentially until the number of combinations surpasses the upper-limit number N_{G}. Furthermore, if the number of combinations surpasses the upper-limit number N_{G}, the data creation processing unit 111 may merge plural combinations with the compressed size at the lowest levels in all combinations.

In the example illustrated in FIG. 17, the compressed size of a combination of “JP and female” is 5000 and the compressed size of a combination of “JP and male” is 7500. Furthermore, the combinations of “JP and female” and “JP and male” are the two combinations with the compressed size at the lowest levels in all combinations. Thus, the data creation processing unit 111 deletes the combinations of “JP and female” and “JP and male” from the group dividing information 210 (see strikethroughs in FIG. 17). The data creation processing unit 111 creates a combination obtained by merging the combinations of “JP and female” and “JP and male” as “(JP and male) or (JP and female)” and adds this combination (see underlined parts in FIG. 17).

“The number of individuals” in the combination of “(JP and male) or (JP and female)” becomes 5000, which is the sum of “the number of individuals” of the combinations of “JP and male” and “JP and female.” In “NULL variant list” in the combination of “(JP and male) or (JP and female),” “(0, 2), (50, 0)” registered in “NULL variant list” of the combinations of “JP and male” and “JP and female” in common is registered. Moreover, “compressed size” in the combination of “(JP and male) or (JP and female)” is figured out as 10000 based on the product of the number of NULL variants included in “NULL variant list” and “the number of individuals” in the combination of “(JP and male) or (JP and female).”

### [B-2] Behavior Example

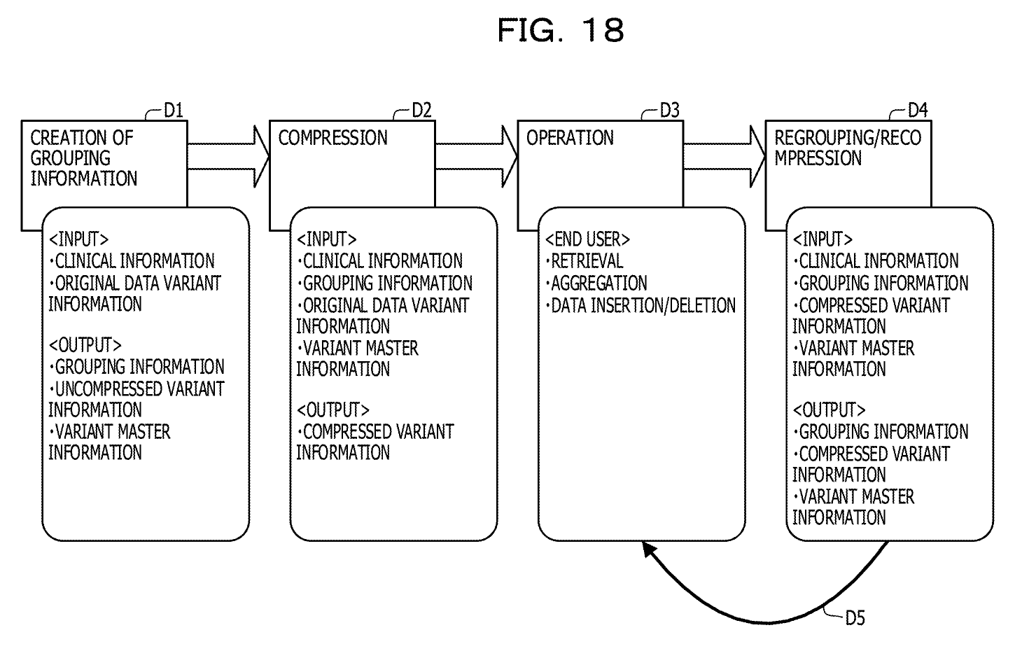

An operation example of variant information in the above-described one example of the embodiment will be described in accordance with a flowchart illustrated in FIG. 18 (processing D1 to D5).

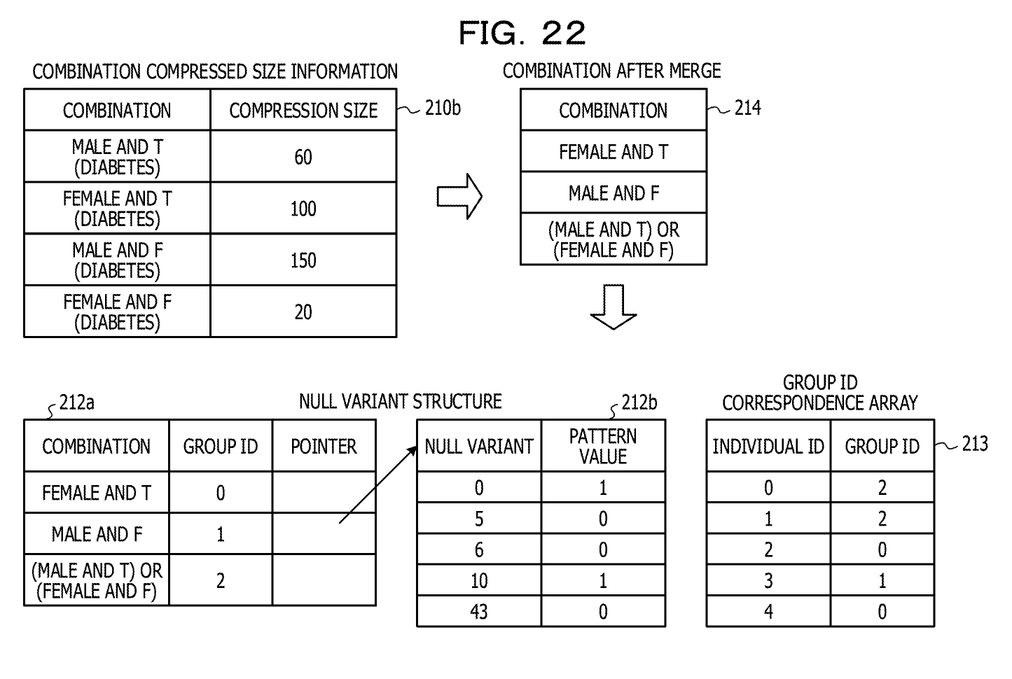

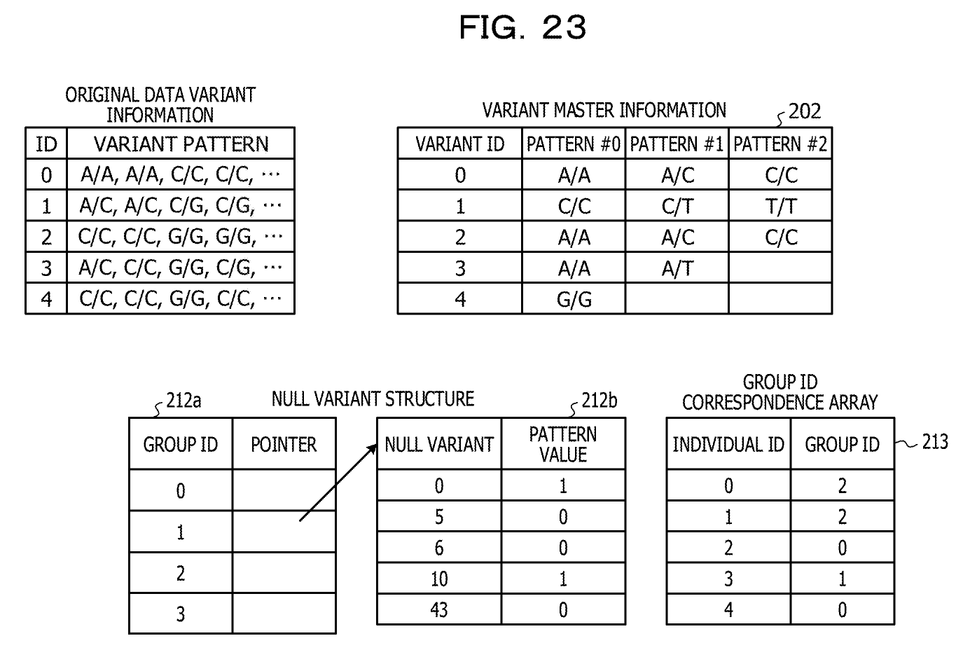

The data creation processing unit 111 creates grouping information (processing D1). For example, the data creation processing unit 111 employs the clinical information 205 and the original data variant information 203 as inputs and outputs the grouping information, the uncompressed variant information 204, and the variant master information 202. The grouping information will be described later by using FIG. 22, FIG. 23, and so forth.

The data creation processing unit 111 executes compression processing of the original data variant information 203 (processing D2). For example, the data creation processing unit 111 employs the clinical information 205, the grouping information, the original data variant information 203, and the variant master information 202 as inputs and outputs the compressed variant information 206.

The aggregation processing unit 112 executes operation processing of the uncompressed variant information 204 (processing D3). The aggregation processing unit 112 carries out retrieval of variants, aggregation of variants, insertion and/or deletion of data based on operation by an end user.

Because the data distribution is changed due to the insertion and deletion of data, the data creation processing unit 111 executes re-creation processing of the group dividing information 210 and recompression processing of the compressed variant information 206 (processing D4). For example, the data creation processing unit 111 employs the clinical information 205, the grouping information, the compressed variant information 206, and the variant master information 202 as inputs. Furthermore, the data creation processing unit 111 outputs the grouping information, the compressed variant information 206, and the variant master information 202.

Thereafter, the processing D3 and D4 are repeatedly executed (processing D5).

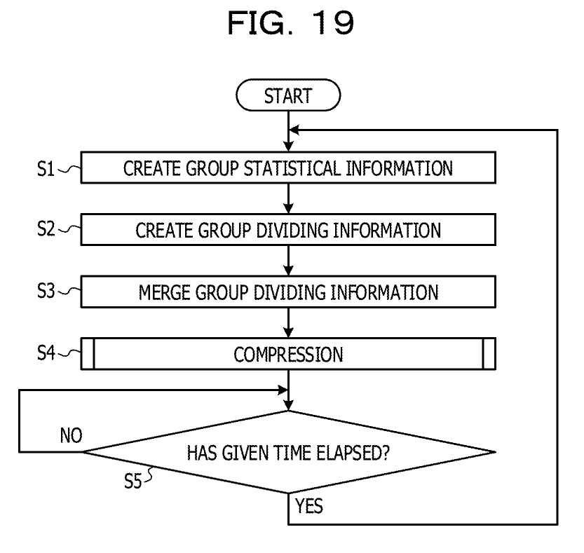

Next, compression processing of the uncompressed variant information 204 in the one example of the embodiment will be described in accordance with a flowchart illustrated in FIG. 19 (S1 to S5).

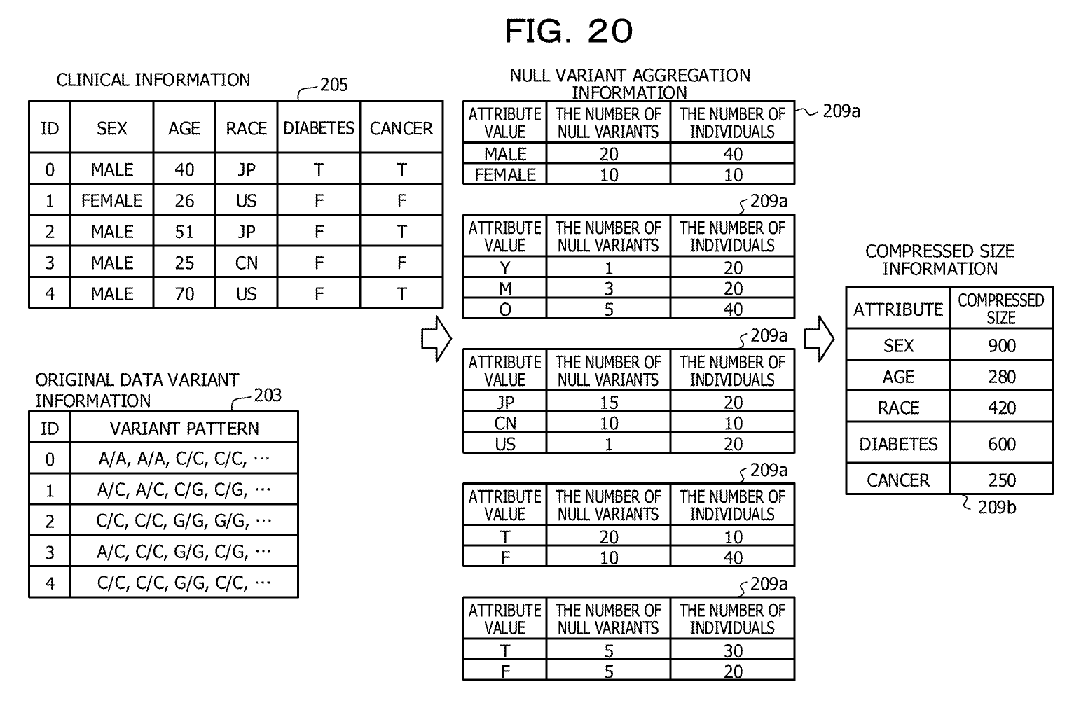

The data creation processing unit 111 creates the group statistical information 209 (S1). Details of the processing of S1 will be described later by using FIG. 20.

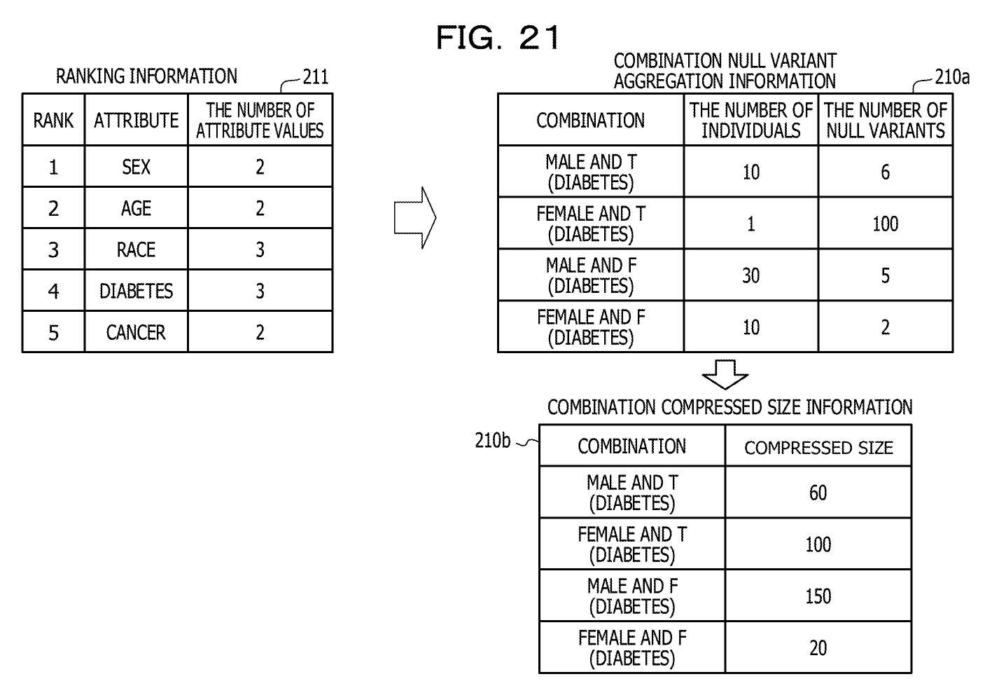

The data creation processing unit 111 creates the group dividing information 210 (S2). Details of the processing of S2 will be described later by using FIG. 21.

The data creation processing unit 111 merges combinations of the created group dividing information 210 (S3). Details of the processing of S3 will be described later by using FIG. 22.

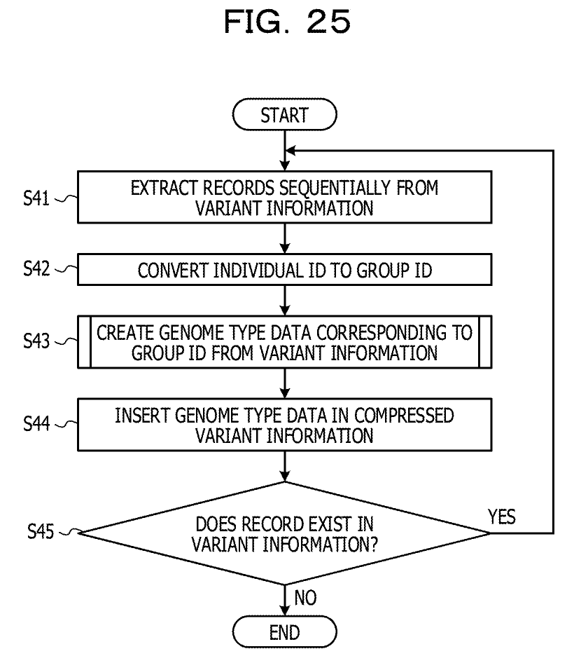

The data creation processing unit 111 executes the compression processing of the uncompressed variant information 204 (S4). Details of the processing of S4 will be described later by using a flowchart of FIG. 25.

The data creation processing unit 111 determines whether a given time has elapsed from the start of the processing of S1 (S5).

If the given time has not elapsed (see No route of S5), the processing of S5 is repeatedly executed.

On the other hand, if the given time has elapsed (see Yes route of S5), the processing returns to S1.

FIG. 20 is a diagram explaining creation processing of compressed size information in one example of the embodiment.

The data creation processing unit 111 creates the NULL variant aggregation information 209a about each attribute included in the clinical information 205 based on the clinical information 205 and the original data variant information 203. In the example illustrated in FIG. 20, five pieces of NULL variant aggregation information 209a about the attributes “sex,” “age,” “race,” “diabetes,” and “cancer” included in the clinical information 205 are created. In the example illustrated in FIG. 20, “attribute value” of the NULL variant aggregation information 209a about the attribute “age” represents Young (Y), Middle (M), and Old (O).

The data creation processing unit 111 creates the compressed size information 209b based on the respective pieces of NULL variant aggregation information 209a. In the compressed size information 209b, the total of the compressed size regarding each attribute is registered.

The NULL variant aggregation information 209a and the compressed size information 209b illustrated in FIG. 20 correspond to the group statistical information 209 illustrated in FIG. 15.

FIG. 21 is a diagram explaining creation processing of combination compressed size information in one example of the embodiment.

The data creation processing unit 111 creates the combination NULL variant aggregation information 210a based on the ranking information 211.

The ranking information 211 represents the ranking of the compressed size in each attribute based on the compressed size information 209b illustrated in FIG. 20. “The number of attribute values” represents the number of attribute values registered in the NULL variant aggregation information 209a about each attribute illustrated in FIG. 20.

In the example illustrated in FIG. 21, the combination NULL variant aggregation information 210a is created regarding the combination of the attributes “sex” and “diabetes.”

The data creation processing unit 111 creates the combination compressed size information 210b based on the combination NULL variant aggregation information 210a. In the combination compressed size information 210b, the product of the number of individuals and the number of NULL variants in each combination is registered.

The combination NULL variant aggregation information 210a and the combination compressed size information 210b illustrated in FIG. 21 correspond to the group dividing information 210 illustrated in FIG. 16.

FIG. 22 is a diagram explaining merge processing of combination compressed size information in one example of the embodiment.

The data creation processing unit 111 merges combinations included in the combination compressed size information 210b in such a manner that the number of combinations included in the combination compressed size information 210b becomes equal to or smaller than the upper-limit value N_{G}. In the example illustrated in FIG. 22, four combinations are registered in the combination compressed size information 210b. Thus, the data creation processing unit 111 merges plural combinations with a small compressed size so that the number of combinations may become equal to or smaller than the upper-limit value N_{G} (for example, 3). In the example illustrated in FIG. 22, the compressed size of “female and F (diabetes)” is 20 and the compressed size of “male and T (diabetes)” is 60. Thus, the compressed sizes are smaller in the combinations included in the combination compressed size information 210b.

Thus, the data creation processing unit 111 merges “female and F (diabetes)” and “male and T (diabetes)” to obtain the combination 214 after merge. In the combination 214 after merge, “female and T,” “male and F,” and “(male and T) or (female and F)” are included.

The data creation processing unit 111 may create the NULL variant structures 212a and 212b and the group ID correspondence array 213 based on the combination 214 after merge. The NULL variant structures 212a and 212b and the group ID correspondence array 213 may be collectively referred to as the grouping information. This grouping information may be used in the compression processing of the uncompressed variant information 204.

In the NULL variant structure 212a, “combination,” “group ID,” and “pointer” are registered in association with each other. “Pointer” refers to the NULL variant structure 212b in which “NULL variant” and “pattern value” about corresponding “combination” are registered. In the NULL variant structures 212a and 212b in FIG. 22, group ID=1 is given to the combination of “male and F” and it is indicated that the NULL variants of this combination are variants #0, #5, #6, #10, and #43. In the NULL variant structure 212b of FIG. 22, it is indicated that NULL variants #0, #5, #6, #10, and #43 about the combination of group ID=1 have the variant patterns of patterns #1, #0, #0, #1, and #0.

The group ID correspondence array 213 represents which “group ID” in the NULL variant structure 212a “ID” of each individual (for example, “individual ID”) in the clinical information 205 illustrated in FIG. 20 corresponds to. In the example illustrated in FIG. 22, for example, individual ID=0 corresponds to group ID=2, and individual ID=1 corresponds to group ID=2, and individual ID=2 corresponds to group ID=0.

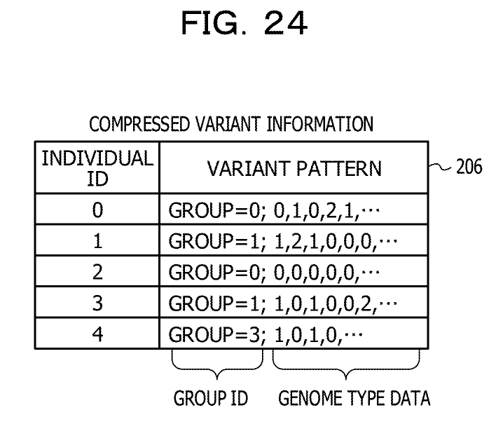

FIG. 23 is a diagram exemplifying input data in compression processing of uncompressed variant information in one example of the embodiment. FIG. 24 is a diagram exemplifying output data in compression processing of uncompressed variant information in one example of the embodiment.

The data creation processing unit 111 creates the compressed variant information 206 illustrated in FIG. 24 based on the original data variant information 203, the variant master information 202, the NULL variant structures 212a and 212b, and the group ID correspondence array 213 illustrated in FIG. 23. In recompression processing, the data creation processing unit 111 may create the compressed variant information 206 illustrated in FIG. 24 based on the uncompressed variant information 204, the variant master information 202, the NULL variant structures 212a and 212b, and the group ID correspondence array 213.

In the compressed variant information 206 illustrated in FIG. 24, “individual ID” and “variant pattern” are associated with each other. In “variant pattern,” the group ID (group) is given to the region previous to genome type data.

Next, details of the compression processing of variant information in the one example of the embodiment will be described in accordance with a flowchart illustrated in FIG. 25 (S41 to S45).

The data creation processing unit 111 extracts records sequentially from the original data variant information 203 (in the case of recompression processing, “uncompressed variant information 204”) (S41).

The data creation processing unit 111 converts the individual ID in the original data variant information 203 (in the case of recompression processing, “uncompressed variant information 204”) to the group ID (S42).

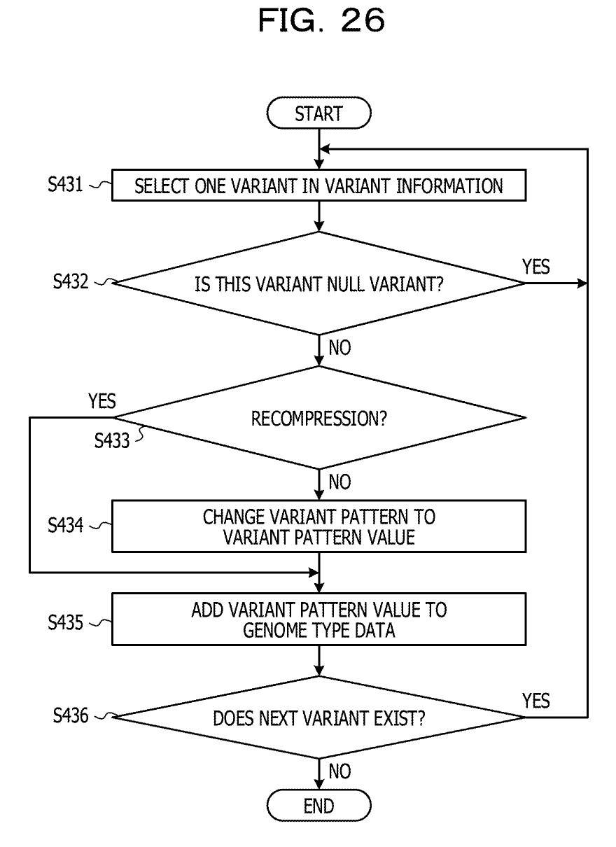

The data creation processing unit 111 creates genome type data corresponding to the group ID from the original data variant information 203 (in the case of recompression processing, “uncompressed variant information 204”) (S43). Details of the processing of S43 will be described later by using a flowchart of FIG. 26.

The data creation processing unit 111 inserts the created genome type data in the compressed variant information 206 (S44).

The data creation processing unit 111 determines whether a record still exists in the original data variant information 203 (in the case of recompression processing, “uncompressed variant information 204”) (S45).

If a record still exists (see Yes route of S45), the processing returns to S41.

On the other hand, if a record does not exist anymore (see No route of S45), the processing ends.

Next, the creation processing of the genome type data in the one example of the embodiment will be described in accordance with a flowchart illustrated in FIG. 26 (S431 to S436).

The data creation processing unit 111 selects one variant in the original data variant information 203 (in the case of recompression processing, “uncompressed variant information 204”) (S431).

The data creation processing unit 111 determines whether this variant is a NULL variant (S432).

If this variant is a NULL variant (see Yes route of S432), the processing returns to S431.

On the other hand, if this variant is not a NULL variant (see No route of S432), the data creation processing unit 111 determines whether the compression processing that is being currently executed is recompression processing (S433).

If the compression processing is recompression processing (see Yes route of S433), the processing proceeds to S435.

On the other hand, if the compression processing is not recompression processing (see No route of S433), the data creation processing unit 111 changes the variant pattern (for example, “ACCT”) to a variant pattern value (for example, “numerical value”) (S434).

The data creation processing unit 111 adds the variant pattern value resulting from the change to the genome type data (S435).

The data creation processing unit 111 determines whether the next variant exists in the original data variant information 203 (in the case of recompression processing, “uncompressed variant information 204”) (S436).

If the next variant exists (see Yes route of S436), the processing returns to S431.

On the other hand, if the next variant does not exist (see No route of S436), the processing ends.

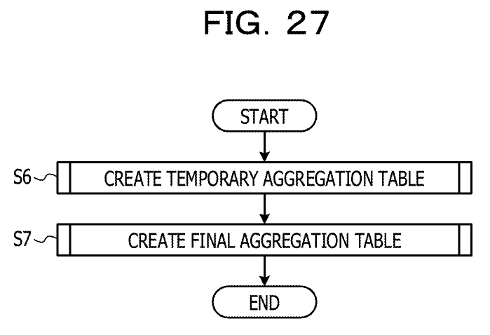

Next, aggregation processing of the compressed variant information 206 in the one example of the embodiment will be described in accordance with a flowchart illustrated in FIG. 27 (S6 and S7).

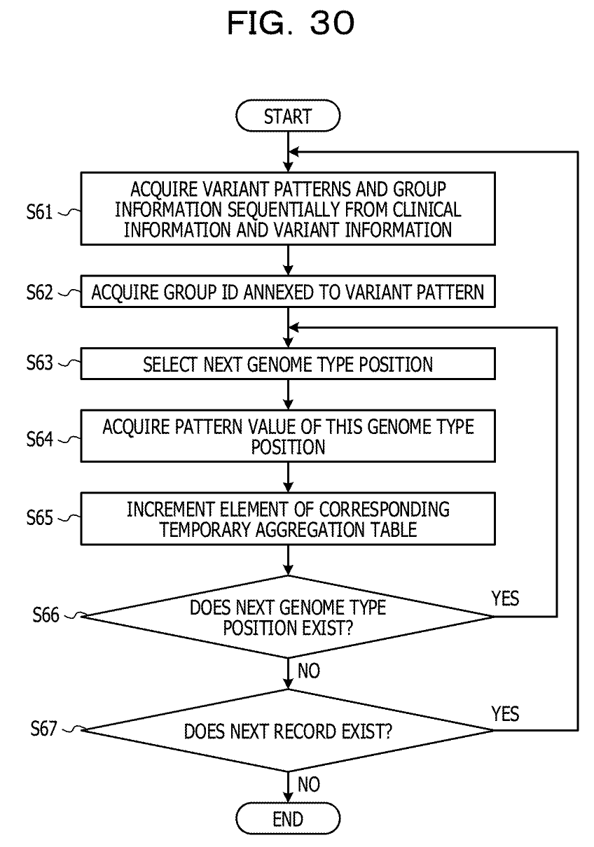

The aggregation processing unit 112 executes creation processing of the temporary aggregation table 207 (S6). Details of the processing of S6 will be described later by using a flowchart of FIG. 30.

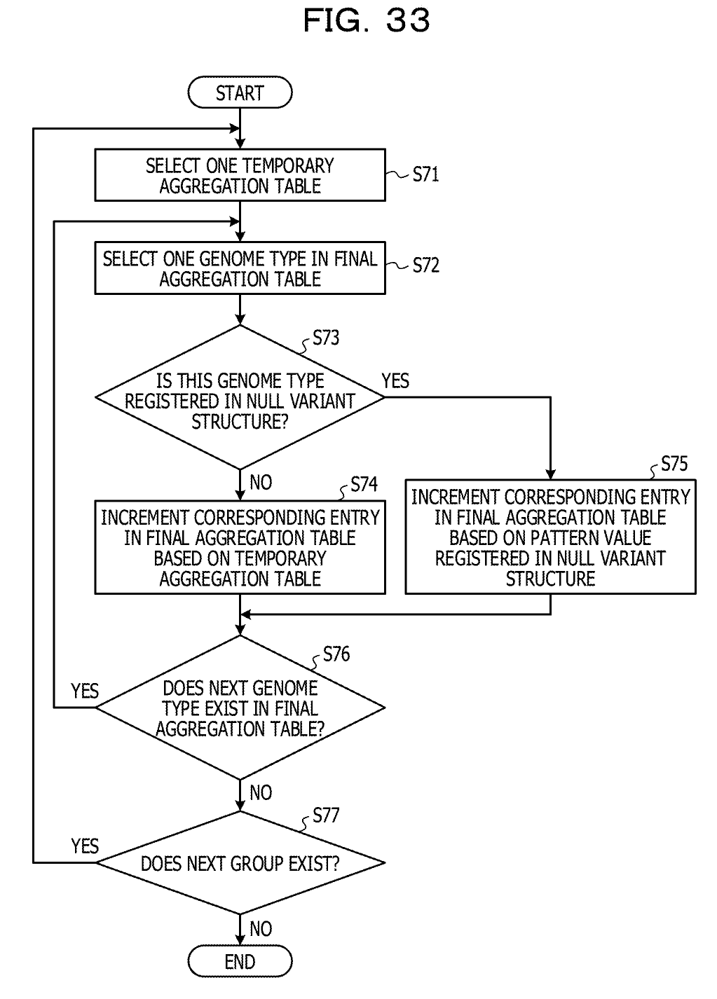

The aggregation processing unit 112 carries out creation of the final aggregation table 208 (S7) and the processing ends. Details of the processing of S7 will be described later by using a flowchart of FIG. 33.

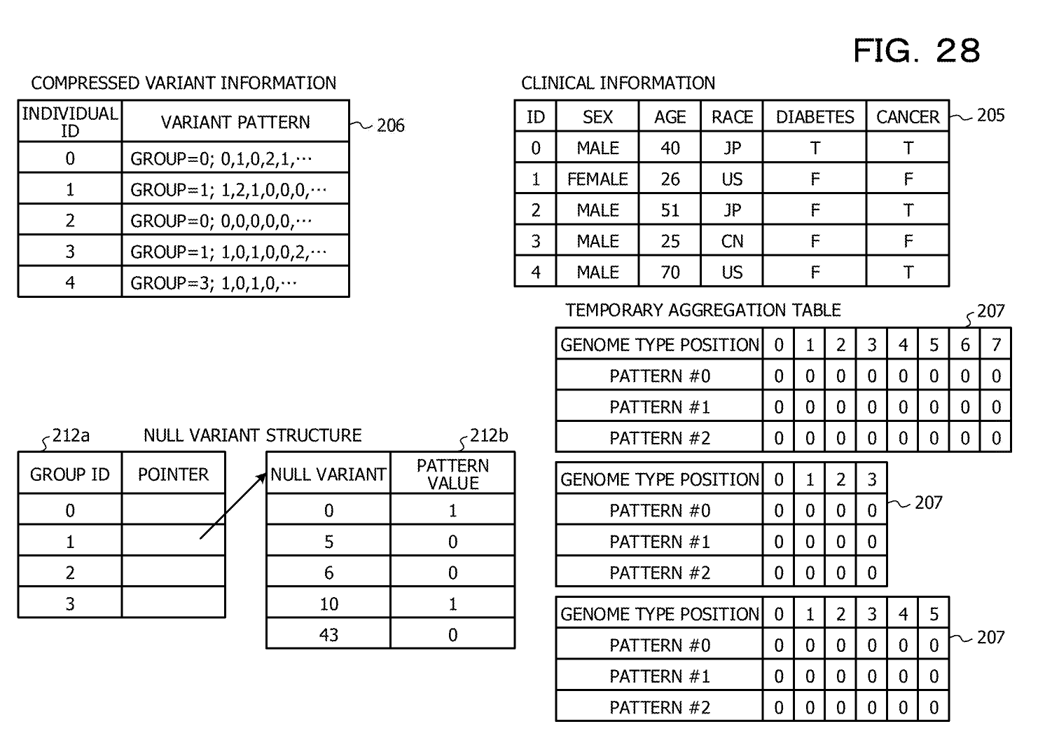

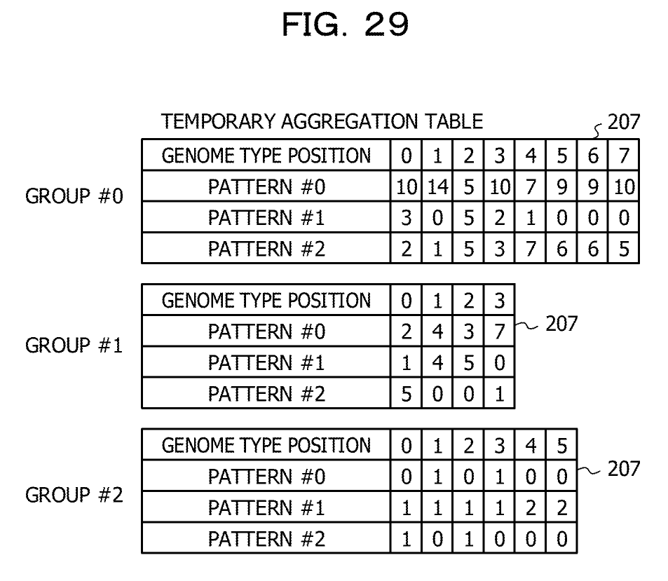

FIG. 28 is a diagram exemplifying input data in creation processing of a temporary aggregation table in one example of the embodiment. FIG. 29 is a diagram exemplifying output data in creation processing of a temporary aggregation table in one example of the embodiment.

The aggregation processing unit 112 creates the temporary aggregation tables 207 illustrated in FIG. 29 based on the compressed variant information 206, the clinical information 205, the NULL variant structures 212a and 212b, and the temporary aggregation tables 207 illustrated in FIG. 28.

The temporary aggregation table 207 is created for each of groups (for example, “Japanese,” “Chinese,” and “American” of the race) and represents what number of which variant patterns exist at each genome type position. Because the NULL variants are omitted in the temporary aggregation tables 207, the number of genome type positions differs for each group. In the temporary aggregation tables 207 used as inputs in FIG. 28, all values are set to 0 as the initial state. Meanwhile, in the output temporary aggregation tables 207 in FIG. 29, values that represent how many variant patterns of patterns #0 to #2 exist at each genome type position are registered.

In the example illustrated in FIG. 29, for example, in the temporary aggregation table 207 of group #0, it is indicated that, at the 0th genome type position, ten variant patterns of pattern #0, three variant patterns of pattern #1, and two variant patterns of pattern #2 exist.

Next, the creation processing of the temporary aggregation table 207 in the one example of the embodiment will be described in accordance with a flowchart illustrated in FIG. 30 (S61 to S67).

The aggregation processing unit 112 acquires the variant patterns and group information sequentially from the clinical information 205 and the compressed variant information 206 (S61). The group information represents whether the group to which the acquired variant pattern belongs to the case group or belongs to the control group.

The aggregation processing unit 112 acquires the group ID annexed to the variant patterns of the compressed variant information 206 (for example, “group=0” in FIG. 24) (S62).

The aggregation processing unit 112 selects the next genome type position (S63).

The aggregation processing unit 112 acquires the pattern value of this genome type position (S64).

The aggregation processing unit 112 increments the element in the temporary aggregation table 207 corresponding to the group information, the group ID, the genome type position, and the pattern ID that are being currently processed (S65).

The aggregation processing unit 112 determines whether the next genome type position exists (S66).

If the next genome type position exists (see Yes route of S66), the processing returns to S63.

On the other hand, if the next genome type position does not exist (see No route of S66), the aggregation processing unit 112 determines whether the next record exists in the compressed variant information 206 (S67).

If the next record exists (see Yes route of S67), the processing returns to S61.

On the other hand, if the next record does not exist (see No route of S67), the processing ends.

FIG. 31 is a diagram exemplifying input data in creation processing of a final aggregation table in one example of the embodiment. FIG. 32 is a diagram exemplifying output data in creation processing of a final aggregation table in one example of the embodiment.

The data creation processing unit 111 creates the final aggregation table 208 illustrated in FIG. 32 based on the temporary aggregation tables 207 of the respective groups and the NULL variant structures 212a and 212b illustrated in FIG. 31.

The final aggregation table 208 illustrated in FIG. 32 represents what number of which variant patterns exist in each variant in all DNA sequences aggregated. In the example illustrated in FIG. 32, it is indicated that, at variant #0, 50 variant patterns of pattern #0, 100 variant patterns of pattern #1, and 50 variant patterns of pattern #2 exist.

Test processing about each variant is executed based on the aggregation result of each variant in the final aggregation table 208 illustrated in FIG. 32. Thereby, the p-value that represents the degree of significant difference is calculated and the ranking of the variant is output based on the p-value. “Test processing” may be a Chi-squared test, Fisher's test, or the like.

Users such as doctors and medical researchers may identify disease-associated genes from variants at higher levels in the ranking that are considered to have a strong relation to disease or the like.

The disease-associated gene is one variant in some cases and is a combination of plural variants in other cases in the variants at higher levels in the ranking. Thus, the disease-associated gene may be identified through execution of aggregation processing with variants of various combinations about plural variants at higher levels in the ranking.

Next, the creation processing of the final aggregation table 208 in the one example of the embodiment will be described in accordance with a flowchart illustrated in FIG. 33 (S71 to S77).

The aggregation processing unit 112 selects one temporary aggregation table 207 (S71).

The aggregation processing unit 112 selects one genome type position registered in the final aggregation table 208 (S72).

The aggregation processing unit 112 determines whether this genome type position is registered in the NULL variant structure 212b (S73).

If this genome type position is not registered (see No route of S73), the aggregation processing unit 112 increments the corresponding entry in the final aggregation table 208 based on the temporary aggregation table 207 (S74) and the processing proceeds to S76.

On the other hand, if this genome type position is registered (see Yes route of S73), the aggregation processing unit 112 increments the corresponding entry in the final aggregation table 208 based on the pattern value registered in the NULL variant structure 212b (S75).

The aggregation processing unit 112 determines whether the next genome type position exists in the final aggregation table 208 (S76).

If the next genome type position exists (see Yes route of S76), the processing returns to S72.

On the other hand, if the next genome type position does not exist (see No route of S76), the aggregation processing unit 112 determines whether the temporary aggregation table 207 about the next group exists (S77).

If the temporary aggregation table 207 about the next group exists (see Yes route of S77), the processing returns to S71.

On the other hand, if the temporary aggregation table 207 about the next group does not exist (see No route of S77), the processing ends.

### [B-3] Effects

If variant patterns at the same variant position have the same value among plural sequences each including plural variant patterns, the data creation processing unit 111 excludes these variant patterns having the same value from the storing target. The memory 12 stores the plural sequences for which the processing of exclusion has been executed by the data creation processing unit 111.

This may reduce the data amount of the variant patterns. Moreover, information on the variant patterns may be all stored in the memory 12 and therefore the speed of aggregation processing of the variant patterns may be increased.

If variant patterns at the same variant position have the same value among plural sequences included in the same group in one or two or more groups in plural sequences, the data creation processing unit 111 executes the processing of exclusion of these variant patterns from the storing target of the memory 12.

This may further reduce the data amount of the variant patterns by using a characteristic of the DNA sequence that there are a large number of variants having the same variant pattern among all members of a group when grouping regarding the DNA sequences is carried out based on the race, sex, age, or the like.

If variant patterns that correspond have the same value among plural sequences included in a first group and a second group in two or more groups, the data creation processing unit 111 executes the processing of exclusion of these variant patterns from the storing target of the memory 12.

This may efficiently exclude the same variant pattern in the plural groups from the storing target of the memory 12.

The data creation processing unit 111 merges plural combinations with a small amount of data reduction in such a manner that the number of combinations of two or more groups becomes equal to or smaller than a given number. Furthermore, if variant patterns that correspond have the same value among plural sequences included in the merged plural combinations, the data creation processing unit 111 executes the processing of exclusion of these variant patterns from the storing target of the memory 12.

This may limit the number of combinations of groups and collectively carry out data compression regarding combinations with a low degree of contribution to data compression. Thus, the data compression may be efficiently carried out.

The memory 12 stores information that represents the position of the variant patterns deemed as the target of the processing of exclusion in the sequences regarding each of one or two or more groups. The data creation processing unit 111 restores the sequences before the execution of the processing of exclusion by inserting the variant patterns deemed as the target of the processing of exclusion into the sequences for which the processing of exclusion has been executed based on the information that represents the position of the variant patterns deemed as the target of the processing of exclusion.

This may execute processing of aggregation, analysis, and so forth of the variant patterns included in the sequences based on the information regarding the compressed variant patterns.

### [C] Others

The disclosed techniques are not limited to the above-described embodiment and may be carried out with various modifications without departing from the gist of the present embodiment. Each configuration and each kind of processing in the present embodiment may be chosen according to need or may be combined as appropriate.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. An information processing apparatus, comprising:
a memory; and
a processor coupled to the memory and configured to
execute processing relating to a plurality of sequences according to a plurality of variant patterns included in each of the plurality of sequences,
wherein the executing the processing relating to the plurality of sequences includes:
when variant patterns at a same variant position are same among the plurality of sequences, executing processing of exclusion of the same variant patterns from the plurality of sequences, and
storing the plurality of sequences for which the processing of exclusion has been executed in the memory.

2. The information processing apparatus according to claim 1, wherein the processor is configured to
execute the processing of exclusion when variant patterns at a same variant position have a same value among a plurality of sequences included in a same group in one or two or more groups in the plurality of sequences.

3. The information processing apparatus according to claim 2, wherein the processor is configured to
execute the processing of exclusion when variant patterns that correspond have a same value among a plurality of sequences included in a first group and a second group in the two or more groups.

4. The information processing apparatus according to claim 3, wherein the processor is configured to
merge a plurality of combinations with a small amount of reduction in an amount of data stored in the memory due to the processing of exclusion in combinations of the two or more groups in such a manner that the number of combinations of the two or more groups becomes equal to or smaller than a given number, and
execute the processing of exclusion when variant patterns that correspond have a same value among a plurality of sequences included in the plurality of merged combinations.

5. The information processing apparatus according to claim 2, wherein
the memory is configured to store information that represents a position of variant patterns deemed as a target of the processing of exclusion in the sequences regarding each of the one or two or more groups, and
the processor is configured to restore sequences before execution of the processing of exclusion by inserting the variant patterns deemed as the target of the processing of exclusion into the sequences for which the processing of exclusion has been executed based on the information.

6. The information processing apparatus according to claim 1, wherein the sequences are base sequences of deoxyribonucleic acid.

7. A non-transitory computer-readable storage medium storing a program that causes a computer to execute a process, the process comprising:
executing processing relating to a plurality of sequences according to a plurality of variant patterns included in each of the plurality of sequences,
wherein the executing the processing relating to the plurality of sequences includes:
when variant patterns at a same variant position are same among the plurality of sequences, executing processing of exclusion of the same variant patterns from the plurality of sequences, and
causing a storing unit to store the plurality of sequences for which the processing of exclusion has been executed.

8. An information processing method executed by a processor included in an information processing apparatus, the information processing method comprising:
executing processing relating to a plurality of sequences according to a plurality of variant patterns included in each of the plurality of sequences,
wherein the executing the processing relating to the plurality of sequences includes:
when variant patterns at a same variant position are same among the plurality of sequences, executing processing of exclusion of the same variant patterns from the plurality of sequences, and
causing a memory to store the plurality of sequences for which the processing of exclusion has been executed.

9. The information processing method according to claim 8, wherein the executing the processing of exclusion includes
executing the processing of exclusion when variant patterns at a same variant position have a same value among a plurality of sequences included in a same group in one or two or more groups in the plurality of sequences.

10. The information processing method according to claim 9, wherein the executing the processing of exclusion includes
executing the processing of exclusion when variant patterns that correspond have a same value among a plurality of sequences included in a first group and a second group in the two or more groups.

11. The information processing method according to claim 10, further comprising
merging a plurality of combinations with a small amount of reduction in an amount of data stored in the memory due to the processing of exclusion in combinations of the two or more groups in such a manner that the number of combinations of the two or more groups becomes equal to or smaller than a given number, and
the executing the processing of exclusion includes
executing the processing of exclusion when variant patterns that correspond have a same value among a plurality of sequences included in the plurality of merged combinations.

12. The information processing method according to claim 9, wherein
the memory is configured to store information that represents a position of variant patterns deemed as a target of the processing of exclusion in the sequences regarding each of the one or two or more groups, and
the information processing method further comprising
restoring sequences before execution of the processing of exclusion by inserting the variant patterns deemed as the target of the processing of exclusion into the sequences for which the processing of exclusion has been executed based on the information.

13. The information processing method according to claim 8,
wherein the sequences are base sequences of deoxyribonucleic acid.
